(19) United States
(12) Patent Application Publication
(10) Pub. No.: US 2003/0026855 A1
(43) Pub. Date: Feb. 6, 2003

(54) Artificial blood fluids and microflow drag reducing factors for enhanced blood circulation

(76) Inventors: Marina V. Kameneva, Pittsburgh, PA (US); Harvey S. Borovetz, Pittsburgh, PA (US); Toby M. Chapman, Pittsburgh, PA (US); Bartley P. Griffith, Pittsburgh, PA (US); Brandon M. Repko, Pittsburgh, PA (US)

Correspondence Address: Allen Bloom, Esq., Dechert Price & Rhoads, P. O. Box 5218, Princeton, NJ, 08543, US

(21) Appl. No.: 10/037,130

(22) Filed: Jan. 2, 2002

Related U.S. Application Data
(63) Continuation of application No. 09/487,194, filed on Jan. 19, 2000, now abandoned.
Application No. 09/487,194 is a continuation-in-part of application No. 09/339,647, filed on Jun. 24, 1999, now abandoned.
Application No. 09/339,647 is a continuation-in-part of application No. 09/150,138, filed on Sep. 9, 1998, now abandoned.

(51) Int. Cl. A61K 35/78

(52) U.S. Cl. 424/725; 424/744

(57) ABSTRACT

The present invention provides improved artificial blood fluids and microflow drag reducing factors for use in such fluids as well as the restoration and/or enhancement of microcirculation and tissue oxygenation. In accordance with preferred embodiments, artificial blood fluids with synthetic or natural oxygen carrying compounds are improved through the inclusion of small amounts of blood soluble microflow drag reducing factors. Microflow drag reducing factors may be combined with physiologically acceptable carriers to form fluids for the restoration and/or enhancement of microcirculation and tissue oxygenation. Physiologically acceptable carriers are preferred as those having a polyethylene glycol adjuvant. The concentration of microflow drag reducing factor is from about 0.1 ppm to about 10,000 ppm by weight of the blood fluid. Certain embodiments feature the employment of certain third and fourth generation dendritic polymers to improve emulsification of artificial blood fluids.

### RELATED APPLICATIONS

This application is a continuation in part application of U.S. Ser. No. 09/339,647 filed Jun. 24, 1999, which is a continuation in part application of U.S. Ser. No. 09/150,138 filed Sep. 9, 1998, the disclosures of both of which are herein incorporated by reference.

### FIELD OF THE INVENTION

The present invention is directed to improved fluids for use as artificial blood as well as to fluids which are useful for the further preparation of artificial blood. The present invention is also directed to improved microflow drag reducing factors for use in such fluids as well as the restoration and/or enhancement of microcirculation and tissue oxygenation. The invention is further directed to methods for the restoration and/or enhancement of microcirculation and perfusion and oxygenation of mammalian tissues through contacting such tissues with artificial blood fluids and microflow drag reducing factors provided herein.

### BACKGROUND OF THE INVENTION

It is now in general medical practice to replace blood volume lost by persons who have been injured, who undergo surgery or who otherwise are in need of blood replenishment. Such blood volume replacement commonly takes the form of transfusion by natural blood or natural blood components collected from donors. 12 to 14 million units of blood are infused annually in the United States. For a number of reasons, however, replacement with artificial blood fluids is greatly desired. The price of approximately $200 per unit for donor blood continues to escalate because of cost associated with screening. There is still a risk of HIV transmission, other dangerous viruses (hepatitis, herpes, adenovirus, etc.), bacterial infection and possible errors in compatibility testing. For some patients, transfusion by natural blood is impossible for religious or other reasons. Convenience issues are also significant, since natural blood fluids require special handling, storage, record keeping and other procedures. In addition, natural blood fluids are not shelf stable.

It is also part of general medical practice to treat impaired blood circulation and low tissue perfusion conditions with various therapies involving, for example, the use of donor blood, blood products, colloidal-crystalloid transfusion fluids including saline and Ringers lactate, vasoactive drugs such as a- and b-adrenergic agonists, ACE-inhibitors and Ca-channel blockers, thrombolytic/clot dissolving agents including tPA and AMI for stroke, diuretics, sympathomrnetics, anticoagulants and blood thinners. Impaired microcirculation and low tissue perfusion has been known to occur as a result of a variety of conditions, including hemorrhage, severe trauma, ischemic heart disease, diabetes, acute myocardial infarction, acute transient cerebral ischemic attack, sickle cell disease and atherosclerosis. These therapies, however, have had mixed results in restoring microcirculation and improving tissue perfusion.

Loss of blood can partially be compensated for by transfusion with plasma “expanders” and there are a number of products available for this purpose. Several crystalloid and colloidal preparations have been developed as plasma substitutes. These include, e.g., several versions of gelatin, albumin, hydroxyethyl starch, polyvinylpyrrolidone and dextran. These products do not possess the oxygen carrying ability of blood, and do not serve most of the other functions provided by natural blood.

The oxygen transporting function of blood can be replaced by two types of artificial blood formulations known heretofore. Hemoglobin-based blood substitutes are known to use hemoglobin obtained from outdated human or animal blood as an oxygen and carbon dioxide carrier. These include certain crosslinked hemoglobin materials, known as HemAssist™, PolyHeme™, and Hemolink™, proposed, respectively, by the Baxter, Nortthield Labs and Hemosol companies. Recombinant hemoglobin has also formed an active part of artificial blood substitutes offered by the Baxter, Biopure and DNX companies. Encapsulated hemoglobin, which is believed to be bovine hemoglobin located internally to small, elongated sheaths, has been suggested for this use by the Enzon Company while polymerized bovine hemoglobin has been offered by the Biopure and Upjohn companies. Thus, hemoglobin-based artificial bloods have been proposed heretofore, each of which relies upon modified hemoglobin suspended or dissolved in a pharmacologically acceptable medium.

U.S. Pat. No. 4,301,144—Iwashita et al., discloses blood substitutes comprising hemoglobin attached to a polymer, wherein the oxygen-carrying ability of the modified hemoglobin is nearly equal to that of the original hemoglobin and the residence time in the circulation is satisfactorily long.

U.S. Pat. No. 4,336,248—Bonhard et al., discloses a blood replacement having a capacity to transport oxygen corresponding approximately to that of free hemoglobin and a blood residence time which is at least about twice that of free hemoglobin, comprising hemoglobin molecules coupled to one another or to another protein by a coupling reagent comprising an aliphatic dialdehyde.

In U.S. Pat. No. 4,598,064, Walder discloses a blood substitute comprising a cross-linked, stroma-free hemoglobin with cross links between alpha chain subunits, soluble in aqueous and physiological fluids and capable of reversibly binding oxygen, and a pharmaceutically acceptable carrier.

Walder, U.S. Pat. No. 4,600,531, discloses a method of producing a cross-linked hemoglobin derivative suitable for use as a blood substitute.

U.S. Pat. No. 4,670,417 B Iwasaki et al., discloses hemoglobin modified in that a poly(alkylene oxide) is bonded thereto by a bond between a terminal group of poly(alkylene oxide) and an amino group of hemoglobin. This modified hemoglobin is an effective oxygen carrier and can be used in blood substitutes.

Schmidt et al., in U.S. Pat. No. 4,698,387, discloses a blood substitute with increased intravasal half-life comprising at least one tetramer of hemoglobin and at least one adduct of a physiologically acceptable macromolecular agent bound to the allosteric binding site of the hemoglobin. A preferred embodiment of the macromolecular agent has a molecular weight of about 400 D to about 500,000 D.

Cerny, in U.S. Pat. No. 4,900,780, discloses a blood substitute comprising the reaction product of a modified starch having a molecular weight of from 60,000 to 450,000 D or a tetronic polyol having a molecular weight of from 1,650 to 27,000 daltons which is a block copolymer formed by the addition of ethylene and propylene oxide units to ethylene diamine, with a stabilized stroma-free hemoglobin which has been converted to an oxy-acid or diketone.

U.S. Pat. No. 5,438,041, Zheng et al., discloses a high hemoglobin content water-in-oil-in-water multiple emulsion for use as a blood substitute having high oxygen exchange activity.

Hoffnan et al., U.S. Pat. No. 5,661,124, shows a blood substitute comprising a recombinantly produced mutant hemoglobin oxygen carrier and a physiologically acceptable molecule that is less diffusible than dextrose including disaccharide.

U.S. Pat. No. 4,439,424 (Ecanow C. and Ecanow B.) relates to whole blood substitutes comprising sodium chloride, urea, phospholipid, distilled water and albumin, said components forming a system which may include stroma free hemoglobin, an appropriate sterol, electrolytes and proteins.

U.S. Pat. No. 4,439,357 to Bunhard et al., discloses a method for preparing highly purified, stroma-free, non-hepatitic hemoglobin solution.

U.S. Pat. No. 4,529,719 in the name of Tye, depicts a stroma-free tense state tetrameric maimmalian hemoglobin covalently crosslinked with a diamide bond-forming moieties.

Bucci et al., in U.S. Pat. No. 4,584,130, discloses stroma-free hemoglobin crosslinked with reagents that mimic 2, 3 diphosphoglycerate and transform stroma-free hemoglobin into a physiologically competent oxygen carrier.

U.S. Pat. No. 4,777,244 to Bonhard et al. discloses a method for preparing a crosslinked hemoglobin of extended shelf life and high oxygen transport capacity.

Feller et al.'s U.S. Pat. No. 4,920,194 discloses a blood substitute consisting essentially of an aqueous medium wherein fragments of sulfated glycosaminoglycans are covalently linked with hemoglobin to form products with oxygen binding property.

European Patent Application 140,640 in the name of Wong discloses a blood substitute comprising chemically coupling hemoglobin with dextran or hydroxyethyl starch. The blood substitute comprises the formula (PS)—X—(HB)—Z, where PS is a polysaccharide; where X is a covalently bonded chemical bridging group; where HB is a hemoglobin residue; and where Z is an oxygen affinity reducing ligand, containing 2 or more phosphate groups.

Certain non-hemoglobin based materials are used as the oxygen transport—active component of proposed artificial blood fluids. Silicone liquids and fluorocarbons are known for their ability to carry oxygen. In the 1960's, Clark and Gollan demonstrated that mice immersed in oxygenated silicone oil (it was found to be extremely toxic) or liquid fluorocarbon could “breathe” in the liquid. It was demonstrated that perfusion using finely emulsified fluorocarbon could maintain rat brain function for several hours. Geyer, Monroe and Taylor demonstrated that finely emulsified fluorocarbon could replace essentially all the blood of rats with the rats surviving and recovering. This exciting demonstration did not immediately lead to clinical application because certain of the components had a long retention time in the reticulo-endothelial system (RES) and therefore could not be used clinically. Extensive development was carried out in Japan by Naito and Yokoyama, resulting in the development in 1976 of Fluosol™-DA 20 suitable for clinical testing. Perfluorocarbons showed a particularly high gas solubility (40-50 ml of oxygen and 100-150 ml of carbon dioxide dissolve in 100 ml of PFCs), and the high stability of the carbon fluorine bond makes them inert. The combination of their excellent gas carrying capacity and their metabolic inertness supported their use as in vivo gas carriers.

Fluosol-DA is a 20% (w/v) mixture of 7 parts of perfluorodecalin and 3 parts perfluoro-tripropylamine, with 2.7% pluronic F68 as an emulsifier and 0.4% of egg yolk phospholipids to form membrane coating on the emulsion. Unfortunately perfluorodecalin cannot be used to form stable emulsion and perfluorotripropylamine, with a T_{½} of 64.7 days, has to be combined to form the stable emulsion. The much shorter retention time of the fluosol-DA 20 than certain other fluorocarbons allowed its use for clinical trial and testing. Because of the high viscosity of the fluorocarbon emulsion at high concentrations, the maximum amount used is generally only about 20%. Since there is no binding functionality like hemoglobin such that oxygen can only be dissolved in fluorocarbon, sufficient oxygen carriage can only take place when the patients are breathing 100% oxygen. Other problems with fluorocarbons include their rapid removal from circulation via respiration, and their retention in the reticuloendothelial system (RES), resulting in RES suppression. This potentially results in lowered resistance to infection. In addition, side effects were observed in some patients due to complement activation caused by the Pluronic™ surfactant used. Infusion of one ml/kg test dose of Fluosol produced an immediate transient and small drop in neutrophil and platelet counts in some patients. Fluosol-DA has to be stored in a frozen state.

A further type of fluorochemical oxygen carrier is based on perfluoroctyl bromide and perfluorodichloroctane. Both types allow the use of higher concentrations of perfluorocarbon. Oxygen™ developed by the Alliance Pharmaceutical Corp., San Diego, is prepared from perfluoroctyl bromide (C₈F₁₇Br) with egg yolk lecithin as the surfactant. Another blood substitute, Oxyfluor™ developed by HemoGen, St. Louis, is based on the perfluoro-dichloroctane (C₈F₁₆C.) with triglyceride and egg yolk lecithin. The observation of side effects when the dose is about 1.8 g PFC/kg means that at least at present, the use of the new improved preparations of PFC-based blood substitutes is limited to a relatively low dosage. Oxygent has been used in Phase II clinical trials in surgical patients breathing 100% oxygen. The use of 0.9 g/kg of Oxygent appears to be able to avoid need for the use of one unit of blood.

A significant advantage of perfluorochernicals for use as oxygen carriers is that they are synthetic materials, which can be chemically produced in large amounts without dependence on donor blood or other biological sources. At present such oxygen carriers are limited by toxicity concerns to a relatively low dosage of 0.9 g/kg for human use. This low dosage is partly because of side effects observed in humans at dosage of 1.8 g/kg. The patients still must breathe 100% oxygen.

The Russian pharmaceutical firm, Perftoran, manufactures an artificial blood substitute with gas-transporting function, which is based on a perfluorocarbon emulsion called Perftoran™.

Further fluorocarbon materials, which may be oxygen carriers, have been reported for use in ocular surgery. Oktain™, chemically known as perfluoro-n-octane, was developed in France by Opsia. The compound is also manufactured by Infinitec in the United States, where it is marketed as Perfluoron™ indicated for use in vitreoretinal surgery and adapted for sale in Europe. Vitreon™, chemically known as perfluorohydrophenanthrene, is manufactured in the United States by Vitrophage for marketing there and in Canada. These materials appear to have been adapted for ocular surgical purposes.

Moore et al., in U.S. Pat. Nos. 5,502,094 and 5,567,765, discloses physiologically acceptable aqueous emulsions of perfluorocarbon ether hydrides having 8 to 12 carbon atoms for use as contrast media for various biological imaging modalities such as nuclear magnetic resonance, 19_{F} imaging, ultrasound, x-ray, and computed tomography, and as oxygen transport agents or “artificial bloods” in the treatment of heart attack, stroke, and other vascular obstructions, as adjuvants to coronary angioplasty and in cancer radiation treatment and chemotherapy.

U.S. Pat. No. 5,262,442—Heldebrant et al., discloses a process for final preparation, prior to administration to a patient, of a frozen oxygen transporting fluorocarbon emulsion, without degrading pharmacologic properties thereof, comprising rapidly thawing a frozen oxygen transporting fluorocarbon emulsion at a temperature above 40° C. and thereafter storing said thawed emulsion in a liquid state for from over eight hours up to 15 days prior to its administration.

Kaufman et al., U.S. Pat. No. 5,171,755, discloses an emulsion comprising a highly fluorinated organic compound, an oil that is not substantially surface active and not significantly water soluble and a surfactant, for use as oxygen transport agents, artificial bloods or red blood cell substitutes.

U.S. Pat. No. 4,931,472—Erner, discloses an artificial blood comprising a formulation of a highly fluorinated triethylenediamine including perfluorotriethylenediamine, undecafluorotriethylenediamine or decafluorotriethylenediamine, or any combination thereof, dispersed in water, and an emulsifying agent, wherein emulsifying agent is a copolymer of propylene oxide and ethylene oxide.

U.S. Pat. No. 4,917,930—McCormick, discloses a gas transfer agent comprising an aqueous dispersion of a perfluoro compound and a surfactant. An object of the invention is to use higher amounts of perfluoro compounds and lower amounts of surfactant, with proportionately improved capacity for gas transfer and therapeutic effect, and proportionately diminished toxicity attributable to the surfactant.

Schmolka, in U.S. Pat. No. 4,395,393, discloses an artificial blood composition comprising a perfluoro chemical, physiological saline and a polyoxybutylene-polyoxyethylene block copolymer emulsifier.

U.S. Pat. No. 4,613,708 (Riess et al.), discloses oxygen-carrying blood substitutes comprising oil-in-water emulsions of branched perfluoroalkylated ethenes.

In U.S. Pat. No. 4,173,654, Scherer et al. discloses an artificial blood substitute comprising a fluorochemical compound, a surfactant, a physiologically acceptable aqueous carrier solution, and effective amounts of osmotic, pH and oncotic agents.

U.S. Pat. No. 3,962,439, Yokoyama et al. discloses a blood substitute comprising oxygen-transferable perfluorocarbon compounds emulsified in a physiologically acceptable aqueous solution such as Ringers solution.

In U.S. Pat. No. 4,186,253, Yokoyama et al. discloses a perflisate for the preservation of an organ for transplantation comprising Ringers solution, albumin, a liquid perfluorocarbon compound, and an emulsifier.

U.S. Pat. No. 4,423,061 (Yokoyama et al.) discloses a perfluorocycloamine emulsion preparation having oxygen carrying ability. Also disclosed is the use of a polymeric nonionic surfactant and a phospholipid as an emulsifying agent, and an isotonizing agent.

U.S. Pat. No. 4,425,347, also to Yokoyama et al., discloses a perfluorobicyclo compound emulsion preparation having oxygen carrying ability. Also disclosed is the use of a polymeric nonionic surfactant and a phospholipid as an emulsifying agent, a plasma extender, and an isotonizing agent.

Sloviter's U.S. Pat. No. 4,423,077, discloses an artificial blood comprising an emulsion of perfluoro compounds and a physiologically acceptable aqueous medium wherein the perfluoro compound particles are coated with adherent lecithin and about 95 % of particles have a diameter less than 0.2 μm.

U.S. Pat. Nos. 4,866,096, 4,956,390, and 4,895,876 to Schweighardt disclose stable aqueous emulsions comprising perfluorochemicals, and in varying embodiments, phospholipid, triglyceride of fatty acids, and aqueous media.

Segall et al., in U.S. Pat. Nos. 5,733,894 and 5,747,071, discloses an artificial plasma-like substance having at least one water soluble polysaccharide oncotic agent selected from the group consisting of high molecular weight hydroxyethyl starch, low molecular weight hydroxyethyl starch, dextran 40 and dextran 70, and albumin which is buffered by lactate. Also disclosed is the supplementation of the plasma-like solution with sodium chloride and certain ions, including calcium, magnesium and potassium.

Runge, U.S. Pat. No. 5,114,932, discloses a blood substitute comprising a physiologically acceptable fluid electrolyte solution, a physiologically acceptable agent capable of increasing the osmolality of the blood substitute to a value greater than normal blood, an oxygen carrying substance, and a sufficient amount of water to achieve the desired osmolality. Also disclosed is the above blood substitute wherein the agent capable of increasing osmolality is a disaccharide and the oxygen carrying agent is perfluorocarbon, synthetic hemoglobin or recombinant hemoglobin.

U.S. Pat. No. 4,987,154, Long, Jr., discloses an emulsion comprising an emulsifying agent, a fluorocarbon and an osmotic agent for adjusting and maintaining the osmolality of the solution. Also disclosed is the above emulsion wherein the osmotic agent is a sugar selected from the group consisting of glucose, mannose, fructose, or combinations thereof.

Visca et al., in U.S. Pat. No. 4,990,283, discloses a microemulsion comprising an aqueous medium, a perfluoropolyether, and a fluorinated surfactant.

U.S. Pat. No. 5,330,681 to Brunetta et al. discloses stable diphase emulsions consisting of perfluoropolyethers having perfluoroalkyl end groups and a conventional surfactant dispersed in a continuous dispersing phase.

Although the use of perfluorochemical oxygen carriers for artificial blood fluid has progressed, their toxicity continues to present a significant problem. Adverse effects were reported from infusion of perfluoro-compounds, including fever, thrombocytopenia and undesirable immune responses. There are additional concerns about long-term effects that such materials may have on the liver and other organs. Negative environmental effects may also occur since perfluorocarbons are highly stable compounds in the environment.

Hemoglobin-based products also possess toxicity concerns. They have been linked to hypertension, thrombocytopenia, activation of the complement and coagulation cascades, renal damage, reticuloendothelial cell blockage and even lethal toxicity. While these adverse effects may usually be diminished through reduction in the concentration of the oxygen carriers in the artificial blood fluids and in the total amount of such materials ultimately employed, this greatly diminishes the benefit from use of the materials in oxygenation of tissues.

Accordingly, it is greatly desired to provide artificial blood fluids which are, at once, highly effective in transporting oxygen to tissues in mammals treated with the fluids, while exhibiting no or diminished toxicity when compared with similar, existing artificial blood fluids of comparable oxygen carrying capacity.

It is greatly desired to provide artificial blood fluids which have improved shelf stability, which are cost effective, which are easy to use, which are safe from transmission of infectious disease and which are acceptable to persons of all social and religious viewpoints.

It is desired to provide blood fluid components or precursor concentrates, which can be reconstituted into an artificial blood for application to patients in need of the same.

It is also desired to provide improved microflow drag reducing factors that may be used in the fluids of the present invention as well as for restoring and/or enhancing microcirculation and/or tissue perfusion and oxygenation.

Other objects will be apparent from review of the present specification and appended claims.

### SUMMARY OF THE INVENTION

It has now been discovered in accordance with certain embodiments of the present invention, that the employment of certain artificial blood fluids can be greatly improved through the inclusion of small amounts of a member or members of the class of chemical and biochemical compositions which are dominated microflow drag reducing factors. It has been discovered that impaired microcirculation and conditions of low tissue perfusion and oxygenation can be improved through the use of the artificial blood fluids and microflow drag reducing factors of the present invention. It has also been discovered that conditions of normal microcirculation and tissue perfusion and oxygenation can be enhanced through the use of the artificial blood fluids and microflow drag reducing factors of the present invention. Microflow drag reducing agents belong to the group of drag reducing agents, which are known per se and are generally of the class of polymers with mechanical properties which enable them to reduce the flow resistance of their solvents.

In accordance with one embodiment, it has now been found that artificial blood fluids which comprise oxygen carrying fluorocarbon, hemoglobin-based or other oxygenating species can enjoy unparalleled effectiveness in use, while greatly diminishing the toxic effects of the oxygen carrier, through incorporation of a water soluble microflow drag reducing agent at the concentration of from about 0.1 part per million (ppm) to about 10,000 ppm, by weight of the artificial blood fluid.

In accordance with another embodiment, such artificial blood fluids preferably comprise a physiologically acceptable carrier, a colloidal-crystalloid containing a polyethylene glycol, at least one perfluorocarbon—based oxygen carrying compound, and at least one microflow drag reducing factor. Such fluids are easily made sterile, are non-pyrogenic and are shelf stable.

The amount of perfluorocarbon can be from about 1 to about 20 grams per deciliter of the fluid. It is preferred that amount of perfluorocarbon be present of from about 2.5 to about 15 grams per deciliter, with from about 5 to about 10 grams per deciliter being more preferred.

Amounts of microflow drag reducing factor present in the artificial blood fluids of the invention are preferably from about 0.1 to 10,000 parts per million by weight, based upon the weight of the fluid. Amounts of from about 1 to about 100 ppm are preferred with amounts of from about 5 to about 50 ppm being more preferred.

The fluids of the invention, especially those having fluorocarbon components, preferably further comprise at least one emulsifier. Such emulsifiers are preferably present in amounts between 0.05 and 5 grams per deciliter. Emulsifier concentrations are from about 0.1 to about 3 grams per deciliter with about 0.5 to about 2 grams per deciliter being preferred. A class of emulsifiers has been identified as being preferred for use in connection with the formulation of artificial blood fluids in accordance with this invention, especially those based upon perfluorocarbons. Such emulsifiers are the class of dendritic polymers, especially those based upon polylysine linked to polyethylene glycol (PEG). Such dendrimers, terminated with perfluorocarbon termini, are thus, preferred for emulsifying perfluorocarbon-containing artificial blood fluids of this invention. Preferred species are the third and fourth generation dendrimers of the foregoing class.

The present invention also provides concentrates useful in the formulation of artificial blood fluids. These concentrates are designed for long-term storage and are, accordingly, considered to be shelf stable. They comprise concentrates of perfluorocarbonbased oxygen carrying compound together with microflow drag reducing factor and surfactant in ratios such that, when diluted for use, they are in correct proportion for the final product. Such concentrates generally comprise from about 50 to about 99.9% by weight of perfluorocarbon together with an amount of microflow drag reducing factor which will be effective in improving the flow properties of the resulting, diluted, artificial blood fluid. The concentrates further preferably comprise a physiologically acceptable colloidal-crystalloid carrier containing a polyethylene glycol.

The invention also provides artificial blood fluids having hemoglobin—based oxygen carriers. Such artificial blood fluids preferably comprise a physiologically acceptable carrier, cofloidal-crystafloid containing a polyethylene glycol, at least one hemoglobin-based oxygen carrying compound, and at least one microflow drag reducing factor. Such hemoglobin-based oxygen carriers are present in an amount of from about 0.1 to about 5 grams per deciliter of the fluid. The fluid further comprises from about 1 to about 10,000 ppm, by weight, of microflow drag reducing agent. For hemoglobin—based artificial blood fluids, amounts of hemoglobin derivatives present in the fluids for application to patients is preferably from 2 to about 4 grams per deciliter with from about 2.5 to about 3.5 grams per deciliter being still more preferred. Other oxygen-carrying moieties may also be used and may substitute for all or part of the hemoglobin derivatives.

For these fluids, amounts of drag reducing agent of from about 1 to 1000 ppm by weight are preferred with from about 5 to about 500 ppm being more preferred. Concentrates usefuil in formulating artificial blood fluids comprising hemoglobin derivatives may also be provided. These comprise from 50 to about 99% by weight of hemoglobin derivative admixed with at least one microflow drag reducing factor in proportions such that ultimate fluids for administration to patients may be formed through dilution.

The present invention further provides fluids useful in treating patients with impaired microcirculation and/or conditions of low tissue perfusion and/or oxygenation. These fluids may also be useful in enhancing normal microcirculation and tissue perfusion and oxygenation. These fluids may include a physiologically acceptable carrier and at least one microflow drag reducing factor. Preferably the fluids include from about 0.1 to about 10,00 ppm, by weight, of microflow drag reducing factor.

### BRIEF DESCRIPTION OF THE DRAWINGS

FIG. 1a is a composite graphic representation, which demonstrates the effect of the injection into a rat of a very small amount of a microflow drag reducing factor (plant-derived polysaccharide) on the hemodynamic parameters (blood pressure and tissue perfusion). One can see a significant increase in the tissue perfusion as much as 45 times along with a decrease in blood pressure.

FIG. 1b is a composite graphic representation of a control experiment. It demonstrates the effect of Sodium Nitroprusside injected into vascular system of an experimental animal at the same hemodynamic parameters as FIG. 1a. A notable decrease in the tissue perfusion along with a decrease in blood pressure can be seen.

FIG. 2 is a composite graphic representation which illustrates the effect of an injection of a small concentration (10⁻⁶ g/ml or 1 ppm) of a microflow drag reducing factor dissolved in blood on capillary blood flow in normal and diabetic rats. Alloxan-induced diabetic microangiopathies represent a generalized disturbance of the microcirculation accompanied by a reduction in blood flow, vascular lesions, decrease in erythrocyte deformability, and a significant decrease in the number of functioning capillaries. As illustrated in FIG. 2, the administration of the microflow drag reducing factor to the blood of diabetic rats caused a dramatic increase in blood flow. The microflow drag reducing factors of the present invention, can be used to treat a variety of circulatory disorders, including hypertension, high blood pressure, and microcirculatory disorders.

FIG. 3 is a composite graphic representation of the distribution of blood pressure in the vascular system in the cases of normotension, hypertension and presence of microflow drag reducing factor in the blood. As seen, the microflow drag reducing factor increases precapillary pressure level through the decrease of pressure drop in the resistive vessels (small arteries and arterioles).

FIG. 4 displays data recorded during an experimental study of the effect of microflow drag reducing factor on outcome in rats with severe hemorrhage. This experimental model of hemorrhagic shock causes a 100% mortality in control animals. Restoration of the lost blood volume with Plasma-Lyte containing a microflow drag reducing factor at the concentration of about 2 ppm led to recovery of animal hemodynamics. No signs of acidosis were observed after three hours following the hemorrhage.

FIG. S is a composite graphic representation on flow of blood mixed with a 5% perfluorocarbon emulsion (Fluosol®) in a circulating loop with a centrifugal (Bio-Medicus) pump, measured before and after addition of a plant-derived microflow drag reducing agent.

FIG. 6 is a composite graphic representation of the flow of blood mixed with a 5% perfluorocarbon emulsion (Fluosol®) in the same mock circulation loop of FIG. 5, measured before and after addition of a plant-derived microflow drag reducing factor.

FIG. 7a is a third generation PEG-dendritc-poly(lysine) hybrid useful as an emulsifying agent in connection with the present invention.

FIG. 7b is a fourth generation PEG-dendritic-poly(lysine) hybrid useful as an emulsifying agent with the present invention.

FIG. 8 is a composite graphic representation of the flow of saline in the same mock circulation loop of FIG. 5, measured before and after addition of okra-derived microflow drag reducing factor at a concentration of about 200 ppm.

FIG. 9 is composite graphic representation of the flow of saline in the same mock circulation loop of FIG. 5, measured before and after addition of aloe vera-derived microflow drag reducing factor.

FIG. 10 is a display of data recorded during an experimental study of the effect of microflow drag reducing factor on the outcome of rats with severe hemorrhage. Restoration of the lost blood volume with Plasma-Lyte led to 100% mortality in control animals. Restoration of the lost blood volume with Plasma-Lyte containing a microflow drag reducing factor at the concentration of about 2 ppm led to recovery of animal hemodynamics. No signs of acidosis were observed after three hours following the hemorrhage.

FIG. 11 is a composite graphic representation which illustrates the effect of an injection of Natto-derived microflow drag reducing factor on the vascular resistance in normal rats.

The present invention provides enhancement in the treatment of patients in need of blood replacement through the provision of improved artificial blood fluids. The discovery that small amounts of microflow drag reducing moieties, chiefly certain polymers and biopolymers, can greatly improve the efficacy of perfluorocarbon or hemoglobin-based artificial blood fluids has given rise to the ability to employ such fluids effectively, while minimizing or removing the problems associated with toxicity shown in prior systems. It is also now possible to employ artificial blood fluids having much less hemoglobin- or perfluorocarbon-based oxygen carriers than heretofore. The ability to employ smaller amounts of the oxygen carrying compounds, surfactants, emulsifiers and other components in artificial blood fluids without losing their effectiveness permits both economy and improved therapeutics.

The artificial blood fluids and microflow drag reducing factors of this invention provide improved therapeutic modalities over prior artificial bloods and offer new clinical opportunities. In particular, it is now believed to be possible to improve the clinical status of patients suffering from tissue underperfusion (associated with diseases such as atherosclerosis or impaired microcirculation) due to diabetes, acute myocardial infarction, acute transient cerebral ischemic attack, ischemic heart disease, sickle cell anemia, and similar conditions. It has been shown that fluids including small amounts of microflow drag reducing factors are capable of significantly decreasing vascular resistance without affecting vascular tone, thereby reducing blood pressure and increasing blood flow in peripheral vessels. It has also been shown that these fluids including small amounts of microflow drag reducing factors can increase tissue perfusion between about 2 and about 6 times. The overall improvement of circulation which attends employment of the artificial blood fluids and other fluids of this invention with a very small or zero concentration of oxygen carrier and emulsifier, gives rise to diverse usefil therapies employing such fluids.

Additionally, the artificial blood fluids of this invention can provide superior perfusion for preservation and maintenance of the finctionality of isolated organs intended for transplantation.

The artificial blood fluids of the present invention preferably include a polyethylene glycol added for protecting natural blood cells from mechanical damage, also are beneficial for use with artificial organs and therapeutic devices such as artificial hearts, cardiac assist devices, heart-lung machines, dialyzers, perfusion devices and the like. It is an ideal fluid for “priming” extracorporeal blood flow devices. The improved circulatory effects, which attend blood fluids of the invention, reduce blood loss-related trauma, shock and other complications. Other benefits from the present invention will be apparent to persons of ordinary skill in the art.

In accordance with some embodiments, the artificial blood fluids of the present invention are preferably based upon emulsions of oxygencarrying perfluorocarbons. Any of the fluorocarbons known to persons of ordinary skill in the art, including all of those discussed supra, may be employed in connection with one or more embodiments of this invention. Other fluorocarbon derivatives and modifications thereof as may be developed hereafter may also find utility in the present invention so long as they function to carry oxygen in a way which can benefit cells in a living mammal or in mammalian tissue. Emulsifiers are present in amounts of from about 0.05 to about 5 grams per deciliter of blood fluid. It is preferred that emulsifiers be present in amounts from about 0.1 to about 3 g/dl with 0.5 to 2 g/dl being more preferred. Thus, it will be understood that fluorocarbon-based oxygen carrying compositions are best defined by what they do. Such compositions include a fluorocarbon compound or compounds in a form such that the same can be added to the blood in the circulatory system of a patient in need of artificial blood. Such compositions include pharmaceutically acceptable carriers, emulsifiers, salts, and other adjuvants as may be deemed necessary or desirable. In any event, such materials are in a form effective for introduction into the circulatory system. Exemplary fluorocarbon—based oxygen carrying moieties include, without limitation, perflurodecalin and/or perfluorotri-n-propylamine. Other oxygen carriers, such as hemoglobin-based, perfluoroalkanes, perfluoro-ethers, etc., can also be employed within the spirit of this invention.

It will be understood that the fluorocarbon derivatives are generally emulsified for use and that persons skilled in the art are well-versed in attaining such emulsions. Conventional emulsifiers include lethicin, polyethylene glycol (PEG), fatty acids, Pluronic type emulsifiers, oleate salts, PEG perfluorcarbons ethers and the like. Other emulsifiers will likely be useful as well.

The artificial blood fluids of the invention, in addition to the fluorocarbon or hemoglobin-based oxygen carrier, and if desired, emulsifier also contain a microflow drag reducing factor. The amount of perfluorocarbon can be from as low as below one to about 20 grams per deciliter of the fluid. This amount of fluorocarbon compound is much lower than is conventionally employed due to the presence of the microflow drag reducing factor. This is believed to be made possible by the ability of the microflow drag reducing factor to facilitate circulation of the blood fluid through the body of a patient receiving it. While not being bound by theory, it is believed that hydrodynamic resistance to blood flow in the cardiovascular system of patients is significantly diminished through inclusion of the microflow drag reducing factor, such that circulation at a given pressure is significantly improved. The result is that the oxygen carrying compositions and fluids, whether the artificial ones comprising the fluids of this invention, or the natural blood of the patient treated with the fluid, are flowing through the microcirculation system much more efficiently and effectively, thus transporting higher amounts of oxygen to peripheral tissues than under normal physiological conditions. Tissue oxygenation is concurrently enhanced.

The class of molecules which are microflow drag reducing factors are preferred for use in the context of this invention. A number of such factors can be employed with the present invention. The microflow drag reducing agent may be, for example, selected from the class of water soluble synthetic high-molecular weight polymers, polysaccharides, and polypeptides derived from plants such as okra and others, algae, gums, polypeptides and polysaccharides derived from bacteria, synthetic polypeptides and polysaccharides, bie polymers derived from fish slimes, sea-water and fresh-water biological growths, ovomucin of egg-whites, biopolymers derived from human or animal blood, blood plasma and blood cells.

In accordance with the present invention, it has been shown that a very special set of microflow drag reducing factors can be derived from plants. These microflow drag reducing factors may be derived from a variety of plants including, without limitation, plants of the Lily and Mallow (Malvaceae) plant families. In one embodiment of the present invention, the microflow drag reducing factor is extracted from aloe vera (Aloe barbadensis miller) of the Lily plant family. In another embodiment, the microflow drag reducing factor is extracted from okra (Abelmoschus esculentus, Hibiscus esculenta-malvaceae, Luffa acutangula) of the Mallow plant family. Both aloe vera and okra produce polysaccharides that exhibit microflow drag reducing properties in accordance with the present invention. These polysaccharides may also be used for growth, protection and healing. These polysaccharides may be extracted and purified for use as microflow drag reducing factors. It is noted, however, that aloe vera-derived microflow drag reducing factors are extremely sensitive to degradation by oxidation and heat. Accordingly, all attempts may be taken to keep aloe vera-derived microflow drag reducing factors in a sealed and cool or cold environment.

In yet another embodiment, the microflow drag reducing factor is derived from Natto, a traditional Japanese food. The microflow drag reducing factor may be extracted from Natto. It is presently believed that the microflow drag reducing factor that may be extraeted from Natto is comprised of polyglutamic acid on the order of molecular weight from about 100 to about 10,000 kD. Polyglutamic acids may include gamnma- and/or alpha-polyglutamic acids. Natto is traditionally produced by the fermentation of soybeans by the bacteria Bacillus subtillis natto. It is presently believed that the extraction of microflow drag reducing factor from Natto requires the separation of the microflow drag reducing factor from both the soybeans and bacteria.

A preferred method of extracting microflow drag reducing factors from plants may now be described. It may be necessary to break up the cellulose matrix of plants in order to extract microflow drag reducing factors. Several methods and combinations of methods are available for breaking up this cellulose matrix including, by way of example only, the following described methods. Plants may be ground in a blender. It is preferred that this method is used when extracting microflow drag reducing factors from the okra plant. The use of a blender is advantageous in that it provides adequate shear forces to tear a plant apart thus better exposing the microflow drag reducing factors for extraction. However, high molecular weight microflow drag reducing factors may be destroyed or their effectiveness decreased due to the harshness of this method. The leaves of plants may be manually filleted. It is preferred that this method is used when extracting microflow drag reducing factors from the aloe vera plant. While this method may be time consuming, the resulting extract may contain less particulate matter as compared to using a blender. A press may also be used to break down the cellulose matrix. A press may be used to forcefully squeeze extracts from plants. Regardless of the method used, large particulate matter may be separated from extracts by filtration through a nylon straining bag. The extract may be further filtered in order to remove small particulate matter as well as breaking up gelatinous clumps of microflow drag reducing factors. It is preferred that the extracts are macrofiltered. Preferably extracts are macrofiltered through screens having mesh sizes from about 8 to about 1000 microns. The macrofiltration screens may be manufactured from various polymeric materials including, by way of example only, polyethylene, nylon, polypropylene and fluorocarbon.

The resulting extracts may be mixed with solvents. It is preferred that viscous extracts are mixed with solvents. The addition of solvents may make it easier to process an extract. A variety of solvents are suitable for use in the present invention including, without limitation, phosphate buffered saline, saline and water. It is well within the skill of those in the art to determine those solvents and amount of solvents suitable for use in the present invention.

Extracts may be centrifuged at low speed in order to remove particulate matter. Extracts may be mixed with solvents before being centrifuged. It is preferred that the extracts are centrifuged at low speeds of about 2,000 to about 4,000 revolutions per minute (rpm). Preferably extracts are centrifuged for about 30 to about 120 minutes. Extracts may be centrifuged as many times as is deemed necessary. It may be necessary to centrifuge several times in order to obtain a reasonably clear supernatant. It is preferred that the drag reducing effectiveness of the resulting supernatant is determined in vitro using an experimental circulation loop as described infra.

The resulting supernatants may be treated with solvents and slowly agitated to precipitate microflow drag reducing factors from the supernatant. The supernatant may be mixed with solvents and slowly agitated to precipitate microflow drag reducing factors. These solvents may include organic solvents. Solvents suitable for use in the present invention include, by way of example only, ethanol, acetone, aqueous solutions of ammonium sulfate and cetylpyridinium chloride. It is preferred that the ethanol is about 70% to about 95% ethanol. It is preferred that the aqueous solution of ammonium sulfate is an aqueous solution of 3.2 molar ammonium sulfate. It is also preferred to use about 100 mg/ml of cetylpyridinium chloride. The resulting precipitate may be collected by macrofiltration and/or centrifugation. The precipitate may be washed with solvents, including organic solvents. It is preferred that the precipitate is washed with about 95% to about 100% ethanol. The precipitate may be allowed to dry. The precipitate may be allowed to dry in a hood overnight. It is preferred that any remaining solvent is evaporated from the precipitate.

The dried precipitate may be mixed with a solvent and allowed to dissolve. It may take several days for the precipitate to dissolve. It is preferred that the precipitate is dissolved in the same type of solvent as initially mixed with the extract. Preferably the precipitate is dissolved in the same volume of solvent as initially mixed with the extract. For example, the precipitate may be dissolved in 1,000 ml of phosphate buffered saline where 1,000 ml of phosphate buffered saline was initially mixed with the extract. After the precipitate is dissolved, the resulting solution may be filtered and/or centrifuged at high speed in order to remove particulate matter. It is preferred the solution is filtered through filters of about 8 to about 1,000 micron mesh. Preferably the solution is centrifuged at about 7,000 to about 20,000 rpm for about 30 to about 120 minutes. It is preferred that the drag reducing effectiveness of the resulting filtrates and/or supernatants are determined in vitro using an experimental circulation loop as described intfra In order to remove residual ethanol soluble impurities, the resulting filtrates and/or supernatants may again be mixed with solvents, including organic solvents, and the microflow drag reducing factors precipitated. The precipitate may be removed and dried. The resulting precipitate may again be dissolved in solvent. Again, it is preferred that the precipitate is dissolved in the same tppe and volume of solvent initially mixed with the extract. The resulting solution may be filtered and/or centrifuged. It is preferred that the drag reducing effectiveness of the resulting filtrates and/or supernatants may be determined in vitro using an experimental circulation loop as described infra.

The resulting filtrates and/or supernatants containing microflow drag reducing factors may be treated with enzymes in order to digest non-polysaccharide material. Enzymes suitable for use in the present invention include, without limitation, proteases including trypsin and chymotrypsin, deoxyribonucleases (DNAases) and ribonucleases (RNAases). After enzyme treatment, it is preferred that the drag reducing effectiveness of the resulting solutions are determined in vitro using an experimental circulation loop as described infra. Dialysis may then be used to remove enzymes from the solutions. Dialysis may also remove low molecular weight impurities from the solutions. It is preferred that the solutions are dialyzed for about 2 to about 12 hours using a membrane having an about 7,000 to about 2,000,000 molecular weight cutoff rating. Other methods may also suitable for removing low molecular weight impurities and digested non-microflow drag reducing factors from the solutions. These methods may include, without limitation, ultrafiltration (cross flow filtration and/or tangential filtration having about 10 kD, 50 kD, 100 kD, 400 kD, 0.01 um, 0.05 um, 0.1 um and 0.2 um retention ratings), microfiltration (stirred cell microfiltration system having about 100 kD to about 10,000 kD retention ratings), size exclusion chromatography and ion exchange chromatography. Dialysis as well as the other methods may be performed many times in order to obtain pure solutions of microflow drag reducing factors. It is preferred that the drag reducing effectiveness of the pure solutions of microflow drag reducing factors are determined in vitro using an experimental circulation loop as described infra.

The microflow drag reducing factors of the pure solutions may be chemically modified by the addition of water soluble moieties to their finctional groups. For purposes of the present invention, these moieties may be defmed by what they do rather than what they are. These moieties are capable of improving the stability of microflow drag reducing factors to shear forces, chemical breakdown (hydrolysis), enzyme action and/or immunorecognition (antigenicity). Modification of microflow drag reducing factors may be accomplished, by way of example only, through the use of coupling agents and components having good leaving groups. Coupling agents suitable for use in the present invention include, by way of example only, dicyclohexyl carbodiimide and 1-[3-(dimethylamino)propyl]-3-ethylcarbodiimide. The leaving groups suitable for use in the present invention include, by way of example only, N-hydroxysuccinimide. It is well within the skill of those in the art to determine the amount of moiety to be added. The amount of moiety may be determined by the moieties effectiveness in protecting microflow drag reducing factors against mechanical and enzymatic degradation as well as its ability to maintain solubility of the microflow drag reducing factor in aqueous solution. It is preferred that the drag reducing effectiveness of chemically modified microflow drag reducing factors are determined in vitro using an experimental circulation loop as described infra The chemically modified microflow drag reducing factors may then be purified. The chemically modified drag reducing factors may be purified by a variety of methods, including without limitation, ultrafiltration, microfiltration, dialysis, size exclusion chromatography and ion exchange chromatography. Purification may include the removal of low molecular weight impurities. It is preferred that the drag reducing effectiveness of the purified chemically modified microflow drag reducing factors are determined in vitro using an experimental circulation loop as described infra.

Certain non-naturally occurring synthetic polymers are useful such as highmolecular weight polyethylene oxides, polyacrylamides, and the like. For example, products with the following tradenames and available from the following companies may be useful in one or more embodiments of the present invention as microflow drag reducing factors. Polyethylene oxides (Polyox water soluble resins WSR-301, 309, N60K, N-750 and others, Union Carbide Co., USA) polyacrylamides (Praestol 2515TR, 2540TR and others, Stockhausen, Inc., Sweden), Carboxymethyl cellulose (Gum Technology Co.), gums such as Gum Guar (Sigma Chemical Co.), Tragacanth (Gum Technology Co.), Gum Karaya (Sigma Chemical Co.), Gum Xanthan (Sigma Chemical Co.).

The microflow drag reducing factors useful in the present invention are best defined by what they do rather than by what they are. Thus, such materials are polymers or biopolymers which are water soluble under conditions suitable for the purposes of this invention. Such polymers must be non-pyrogenic, capable of acceptable shelf stability and consistent with use as a component in the circulation of a maimmal. A major requirement is that compound be capable of reducing microcirculatory blood flow resistance generated by vessel bifurcations, constrictions, expansions, and other local changes in the vessel geometry as well as by the chaotic motion of blood cells. Persons of skill in the art will readily be able to identify subclasses and individual compounds belonging to the class of microflow drag reducing factors.

The drag reducing effectiveness of microflow drag reducing factors and microflow drag reducing formulations may be determined. For purposes of the present invention, drag reducing effectiveness may be defined as an increase in flow rate for a certain pressure or a decrease in the pressure required to achieve a certain flow rate. Drag reducing effectiveness may be determined in vitro. An experimental circulating loop may be used to determine the drag reducing effectiveness of microflow drag reducing factors. The experimental circulating loop may have a developed turbulent flow regime. The experimental circulating loop may comprise a centrigal pump (Biomedicus Inc.), an inline flowmeter (Biomedicus Inc.), a pressure transducer (Baxter Health Corp.), a pressure monitor (Alpha Space Labs), ⅜ inch Tygon tubing, a small diameter glass tube about 0.49 centimeters in diameter and about 90 centimeters in length, and a reservoir. The small diameter glass tube may provide resistance in the experimental circulating loop. A microflow drag reducing factor or a microflow drag reducing formulation may be added to saline flowing at a rate of 3.7 liters per minute through the experimental circulating loop and any pressure gradient reduction measured. Drag reducing effectiveness of may be demonstrated by an increase in flow rate for a certain pressure or a decrease in the pressure required to achieve a certain flow rate.

The ability of certain water soluble linear macromolecules to increase fluidity of blood has been studied since 1970. It has been shown that at very low concentrations in blood, these agents were effective in reducing “friction” in the turbulent flow of blood in vitro. Green, H. L. et al., Symposium on Flow, Pittsburgh, 1971; Green, H. L., et al,. Biorheology, 7(4): 221-223 (1971); Stein, P. D., et al., Med. Res. Eng. September-October 6-10 (1972); Green, H. L., et al., Flow, Its Measurement and Control in Science and Industry in Dowdell, R. B., ed., Ann Arbor, Mich. (1974). This effect was specifically referred to as a drag reduction phenomenon (the “Toms Effect”) discovered in 1947. Toms, B. A., Proc 1^{st} Int. Congr. Rheology, 2, Amsterdam (1948). It is believed that under conditions of turbulent pipe flow, dilute solutions of certain polymers require much less energy expenditure for a particular flow rate than that required for the pure solvent.

U.S. Pat. No. 4,154,822 discloses the administration of a polysaccharide derivative from okra plants, which causes hemodynamic and rheologlcal effects which enhance cardiac output The mechanism was said to be a reduction in blood viscosity at relatively low shear rates, due to administration of the polysaccharide to the blood.

The effect of drag reducing polymers on blood circulation in vivo cannot be explained solely by the Toms effect, however, since blood flow in the cardiovascular system is not turbulent. Neither can it be explained by reduction in blood viscosity as suggested by Polimeni et al. in the U.S. Pat. No. 4,154,822, since the reduction in low shear blood viscosity can only be due to a decrease in the red blood cell aggregation. No other materials causing reduction in red blood cell aggregation produce hemodynamic effects.

Vascular flow drag reducing phenomenon.

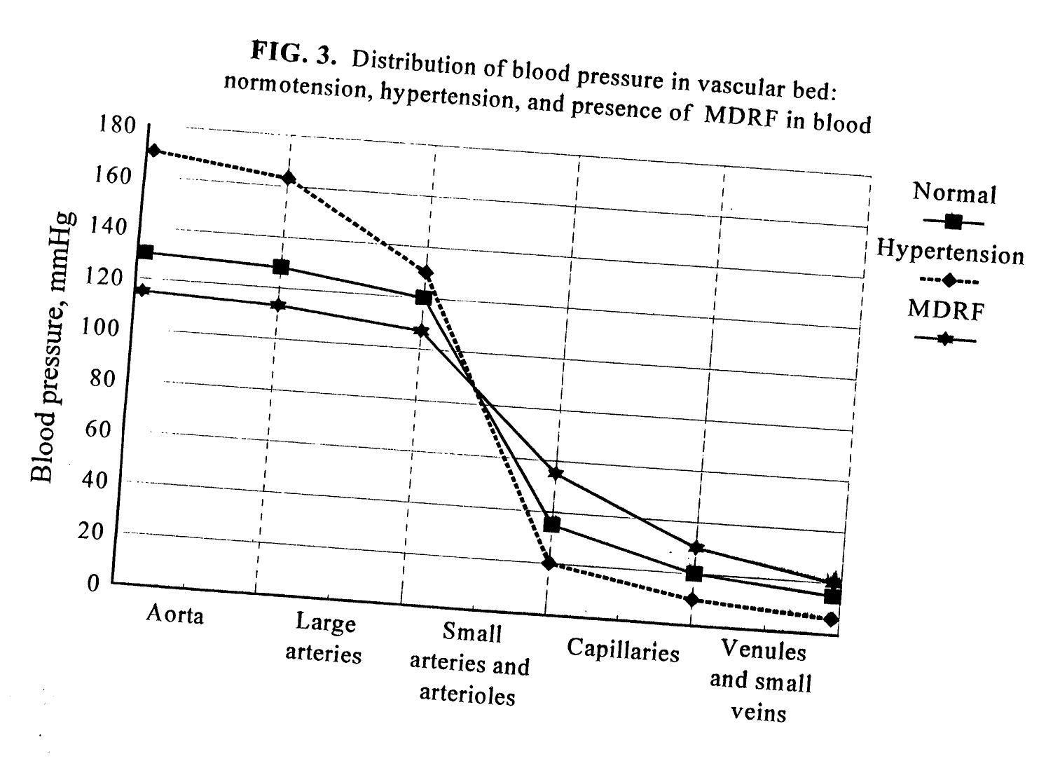

Certain new aspects of drag reducing compounds have been investigated by one of the present inventors through in vitro experiments performed in models of the vascular system. It was found that certain water-soluble high molecular weight linear polymers delay and reduce the development of stagnation zones and eddies at vessel bifurcations, constrictions, expansions and other local changes in vessel geometry, see Kameneva, M. V. et al., Proceeding of the Academy of Sciences of the USSR, Biophysics Section, Jan.-Jun. 22-24, 1988; and Kameneva, M. V. et al., Fluid Dynamics (1990) 25, 6:956-959. This, in turn, causes significant decrease in the blood pressure drop that occurs in the resistive vessels (small arteries and arterioles) increasing precapillary blood pressure and microcirculatory flow (see, e.g., FIG.3). The net result is a reduction in the total arterial pressure as a regulatory response to the decreased total peripheral resistance and increased ricrocirculation.

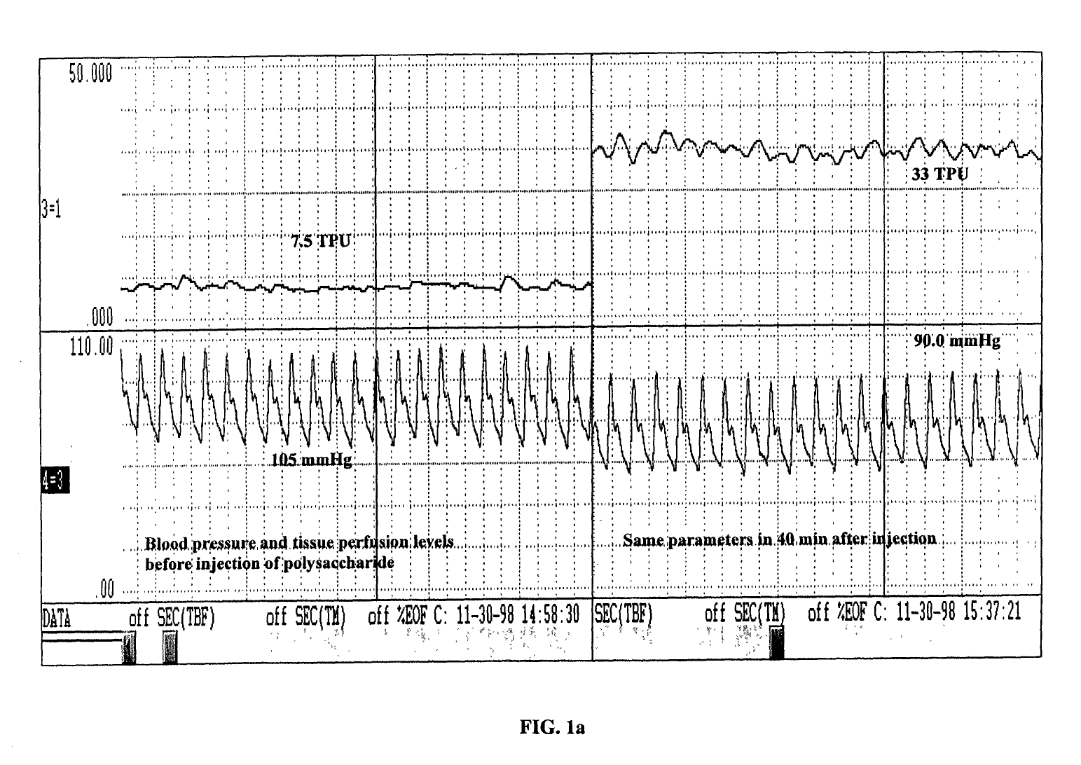

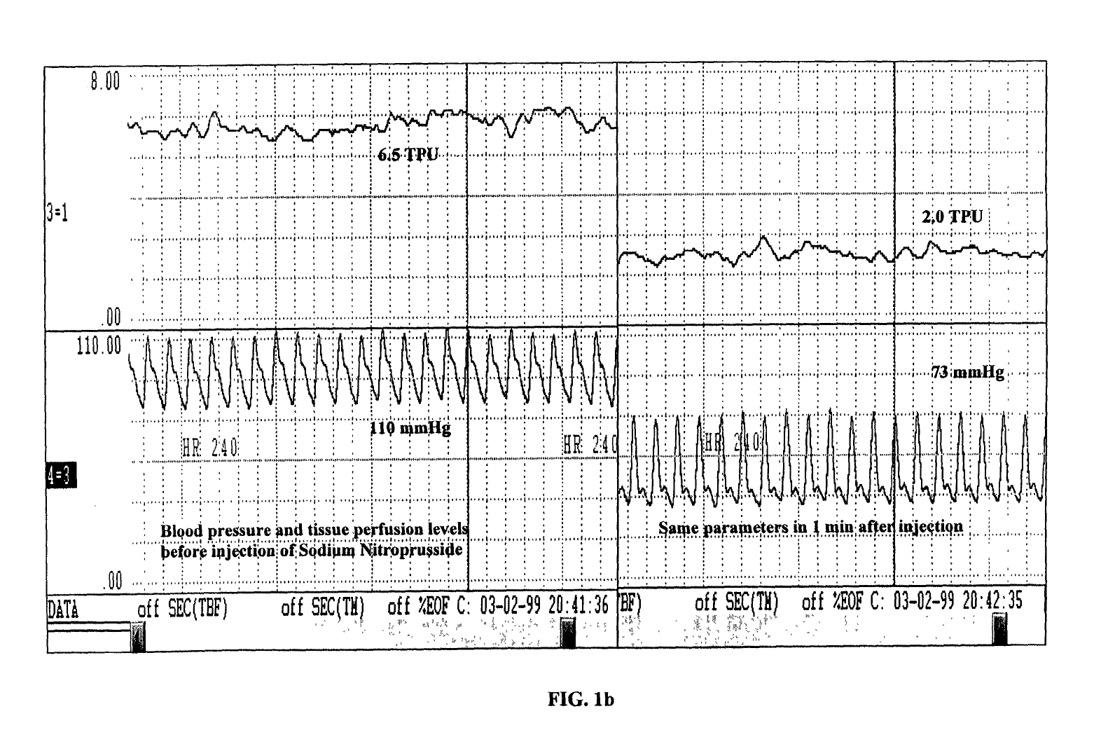

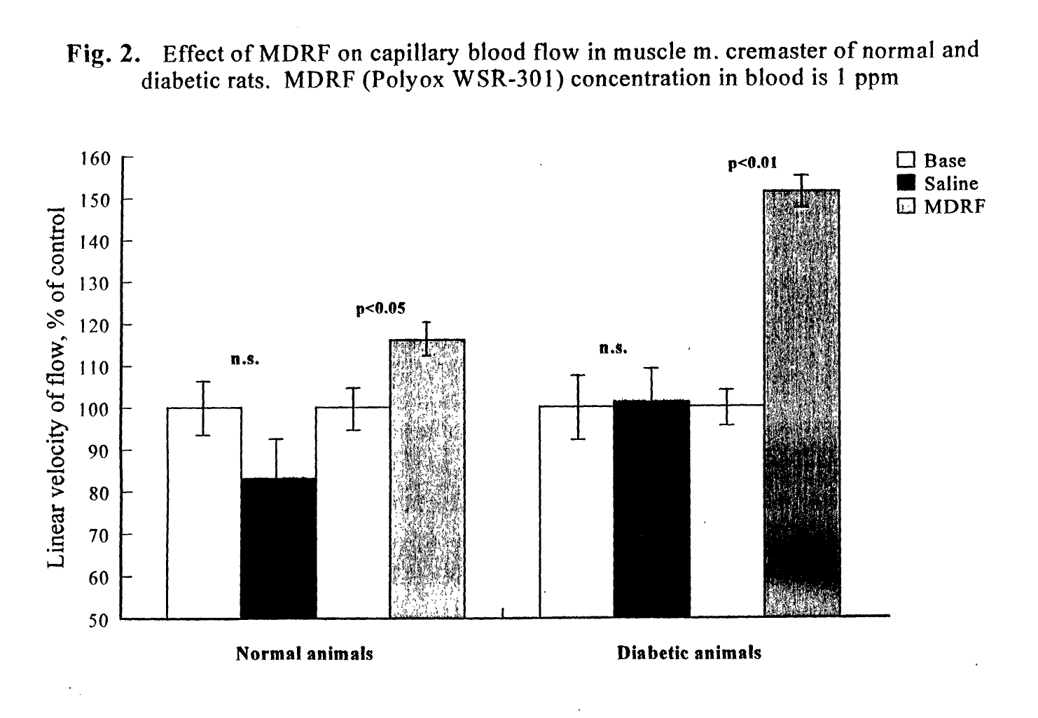

It has been shown that very small amounts of drag reducing polymers introduced into the blood stream in vivo can significantly decrease vascular resistance without decreasing vascular tone, thereby reducing blood pressure and increasing blood flow in peripheral vessels. See Grigorian, S. S., Kameneva, M. V. et al., Soviet Physics.—Doklady 21, 12:702-703 (1976); Grigorian, S. S., Kameneva, M. V. et al., Soviet Physics.—Doklady 23, 7:463-464 (1978); Mostardi, R. A. et al., Biorheology 15(1) 1-14 (1978); Grigorian, S. S. and Kameneva, M. V., Resistance Reducing Polymers in the Blood Circulation in Contemporary Problems of Biomechanics, 99-110, Chernyl, G. G. & Regirer, S. A., eds. (1990), each of which is incorporated herein by reference. Recently, it was shown that a very small amount of a plant derived compound, injected into the vascular system of an experimental animal increased tissue perfusion as much as 2-6 times. This effect was not caused by vasodilatation (FIG. 1a and FIG. 1b).

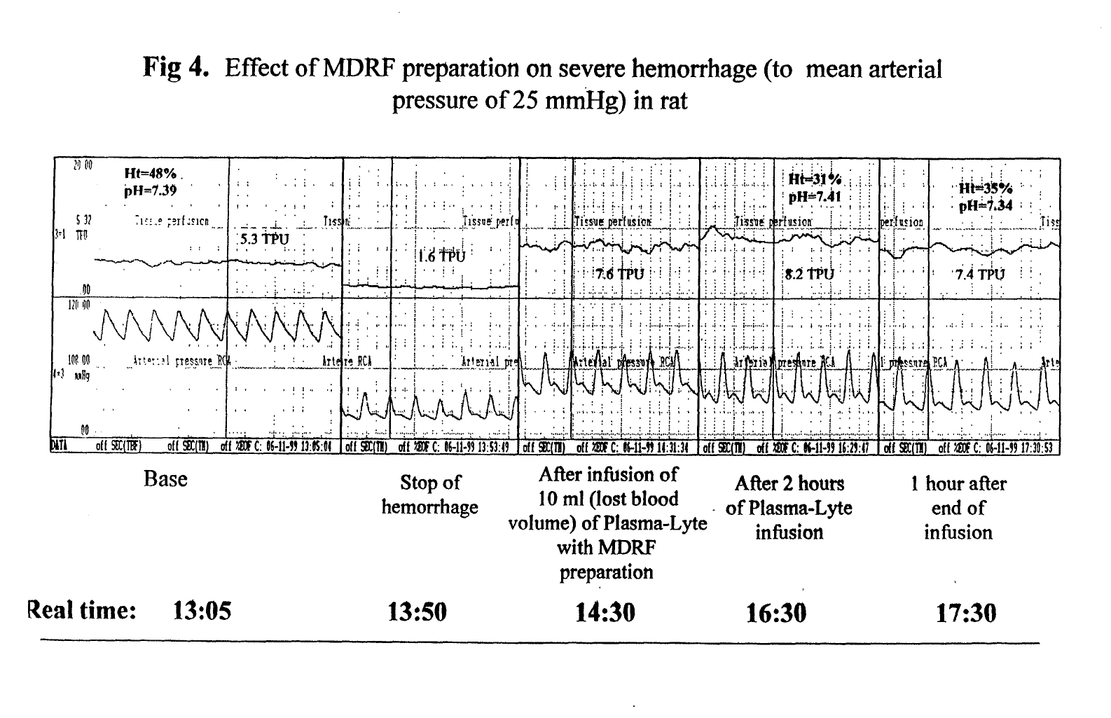

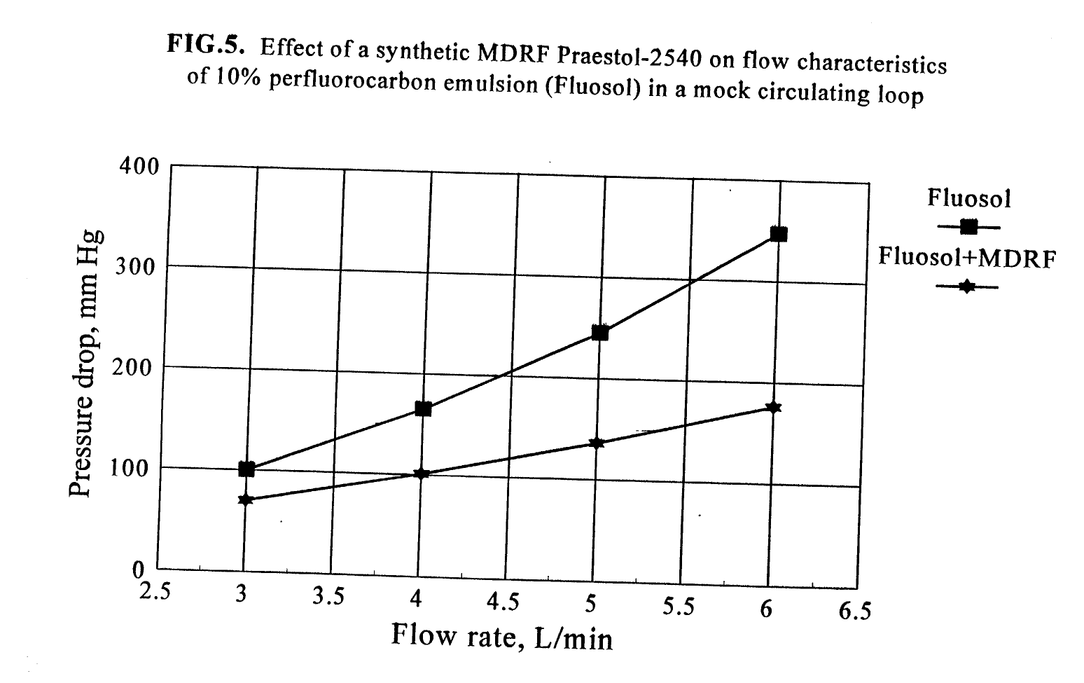

Thus, unlike the Toms Effect, which was associated with turbulent flow conditions, microflow drag reduction occurs at very low flow conditions and may be attributed to the diminishing of local disturbances of flow produced by the geometrical peculiarities of vascular bed and micro-vortices caused by chaotic motion of blood cells. Therefore, the polymers which produce a very strong drag reduction at turbulent flow conditions do not necessarily have the same effect under microflow conditions and vice versa. To evaluate the microflow drag reducing effectiveness of a candidate material, a simple test can be applied for the condition of turbulent flow. See FIGS. 4 and 5. However, for more accurate evaluation of the microflow drag reducing capability of the candidate material, further animal or special hydrodynamic testing (see above) may need to be employed.

Use of microflow drag reducing factors allows the concentration of the oxygen carrier in certain artificial blood fluids to be reduced to levels which previously would have been ineffective, but which provide acceptable oxygen levels of patients consistent with acceptable levels of toxicity to the patients. The oxygen delivery (D) to the organs and tissues can be expressed through the formula D=F•C, where F is a volumetric blood flow rate and C is the concentration of oxygen per volume unit. Thus, an increase in blood flow caused through improved microcirculation allows a corresponding reduction in the concentration of oxygen carrier used in the artificial blood formulation. According to the present invention, perfluorochemical oxygen carriers, previously employed for example, in quantities as high as 20-40 g/dl (20-40% emulsions) or higher, may now be reduced for example, to 1-10 g/dl or even lower, when used with microflow drag reducing factors, while achieving acceptable rates of oxygen delivery. The similar reduction of hemoglobin-based oxygen carriers can be also achieved. Moreover, the artificial blood fluids of this invention with a very small or zero concentration of oxygen carrier and/or emulsifier gives rise to different useful therapies employing such fluids. For example, these fluids can be used for increasing the effectiveness of drug delivery to target organs and tissues utilizing a much lower concentration of the drug.

It is possible to use artificial blood fluids and microflow drag reducing factors of this invention for a much wider range of clinical interventions than possible with prior materials. Such fluids may be used in elective surgery, traumatic injury involving disturbance of microcirculation as well as in cases of significant blood loss, hemorrhagic shock, circulatory shock, medical conditions such as sickle cell anemia, diabetes, acute myocardial infarction, acute transient cerebral ischemic attack, ischemic heart disease and similar conditions. The overall microcirculatory improvement which attends employment of the artificial blood fluids and microflow drag reducing factors of this invention gives rise to diverse, useftl therapies employing such fluids.

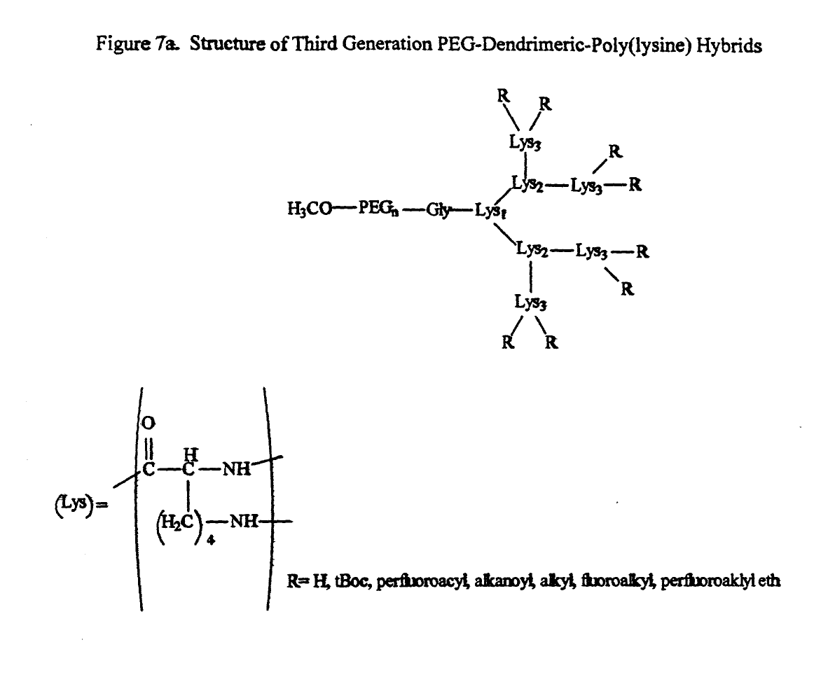

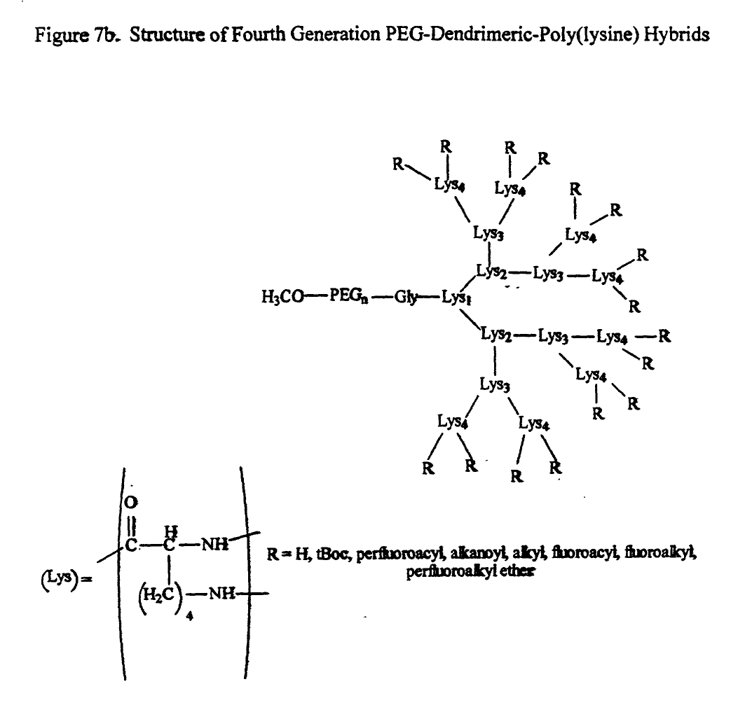

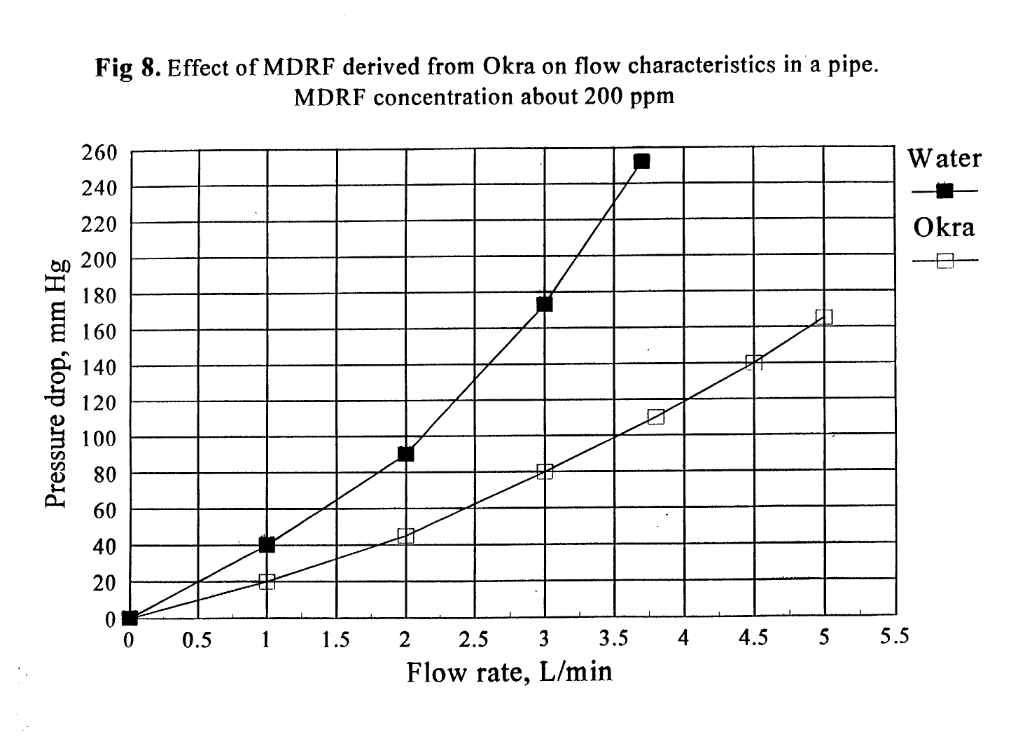

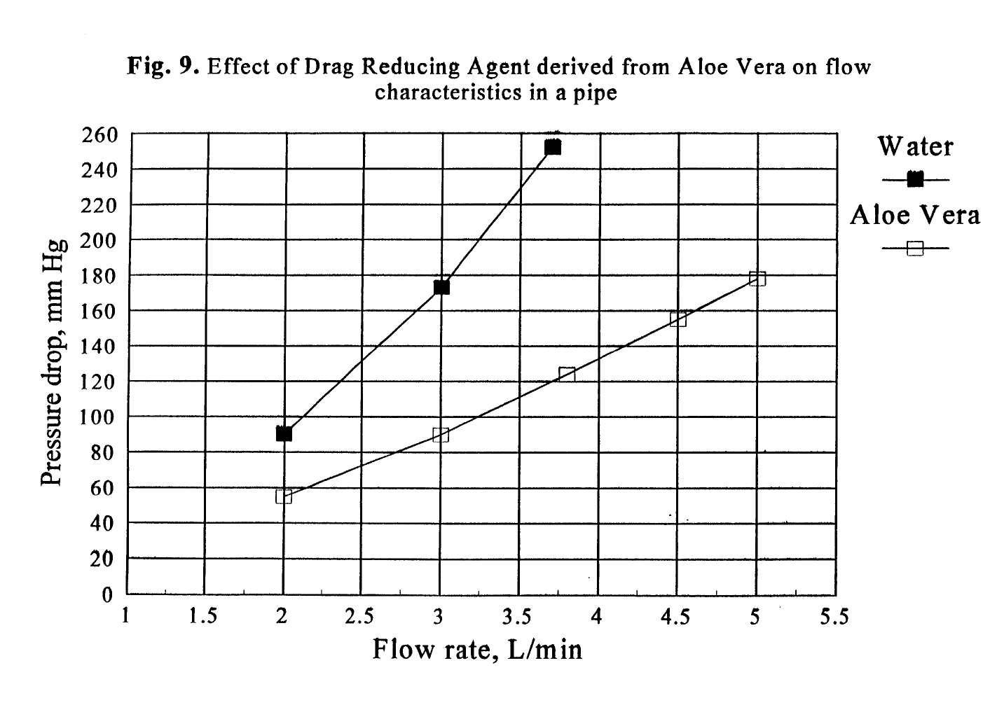

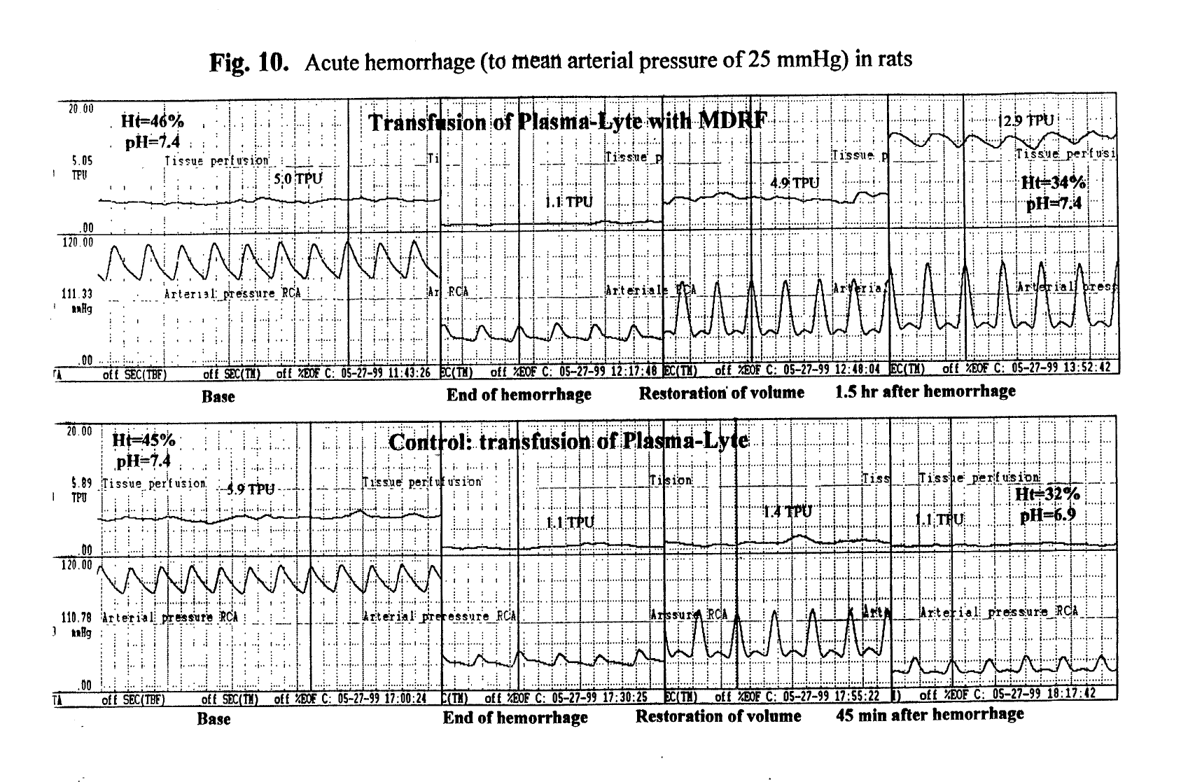

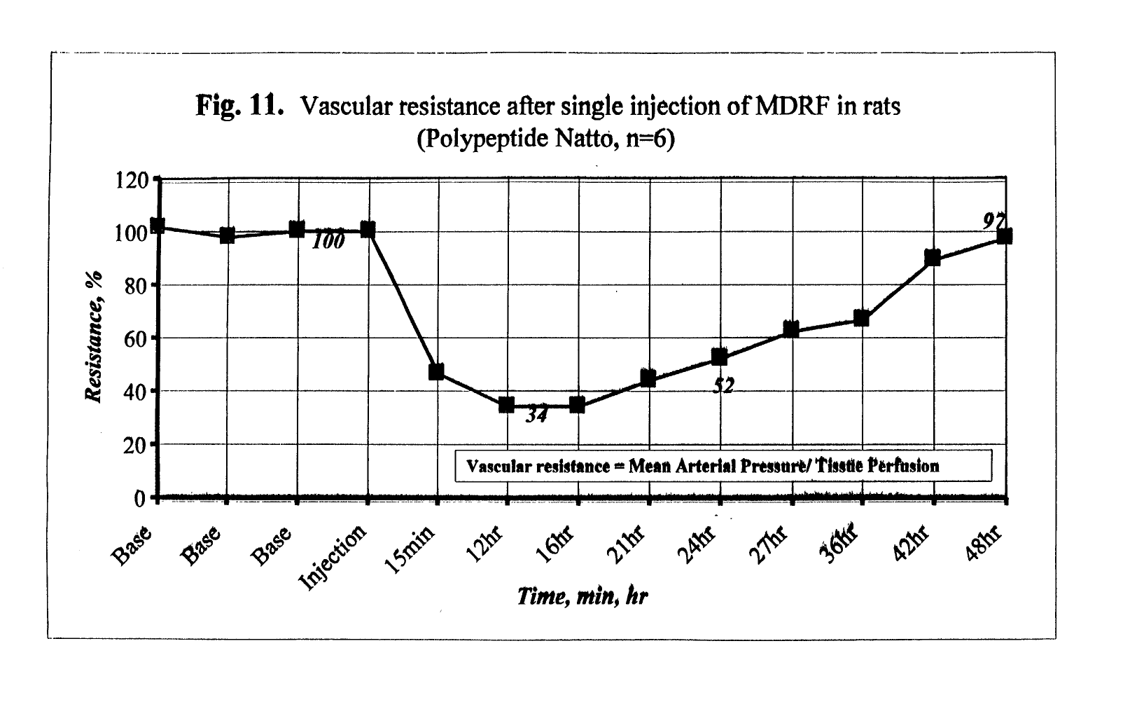

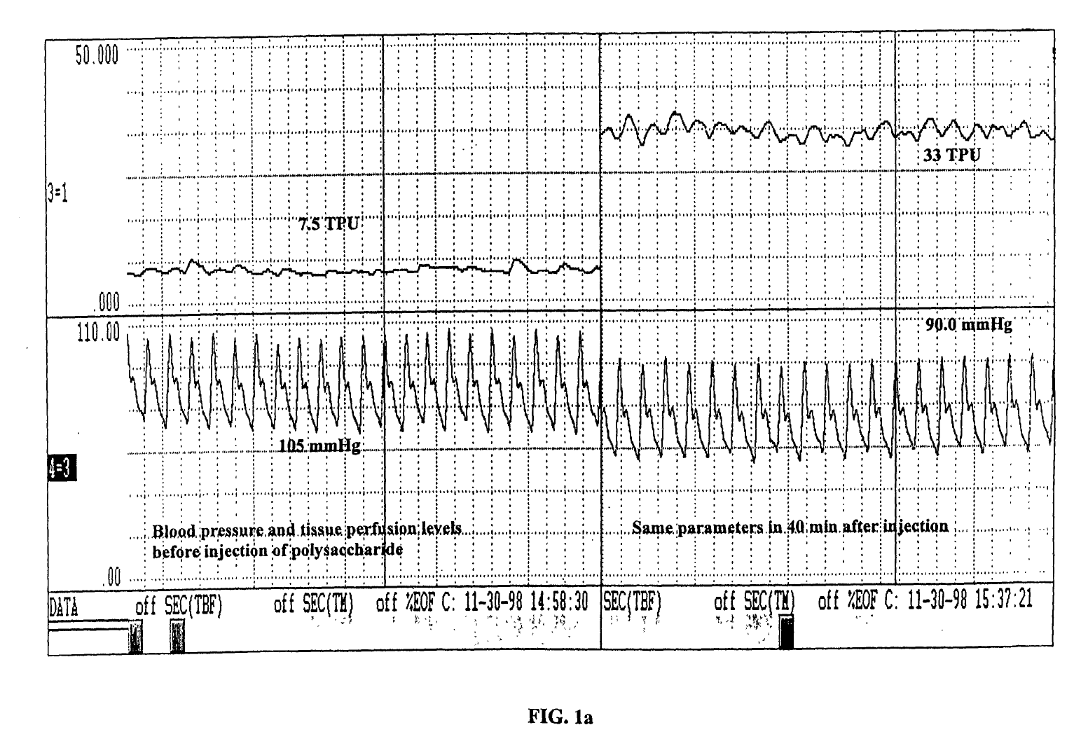

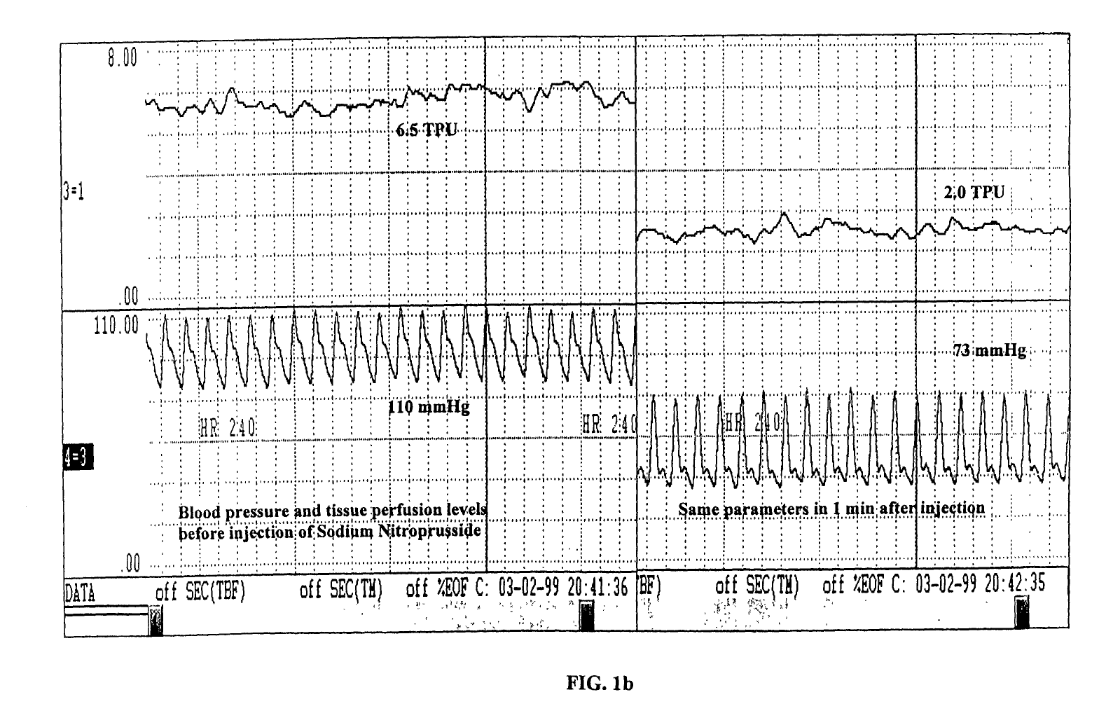

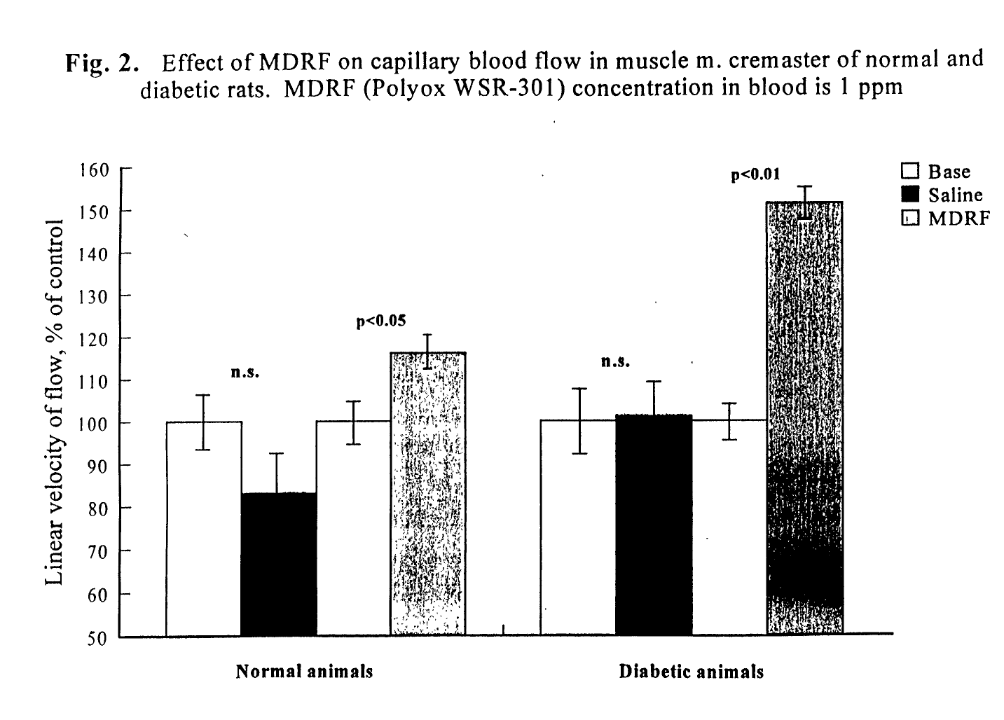

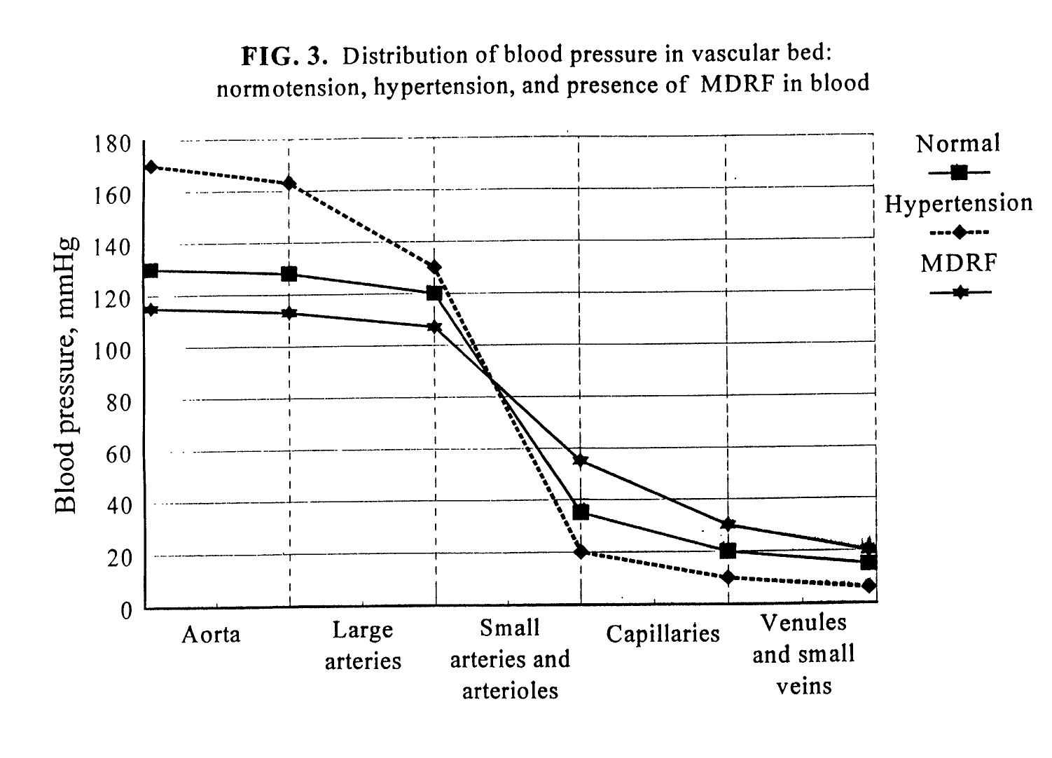

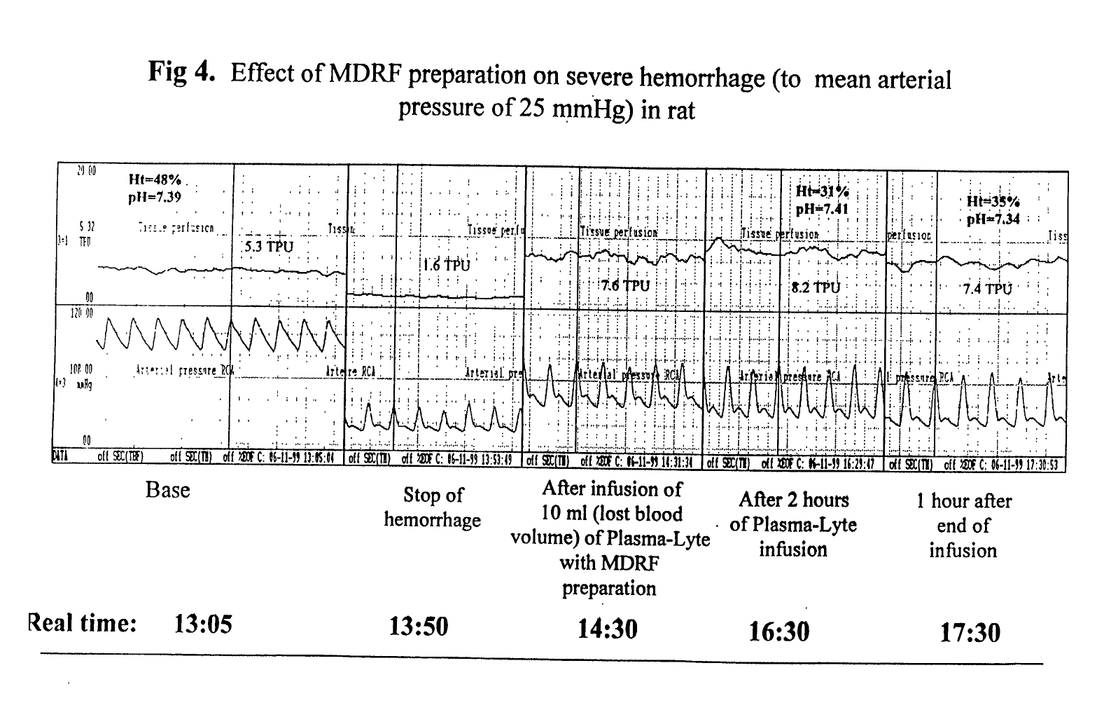

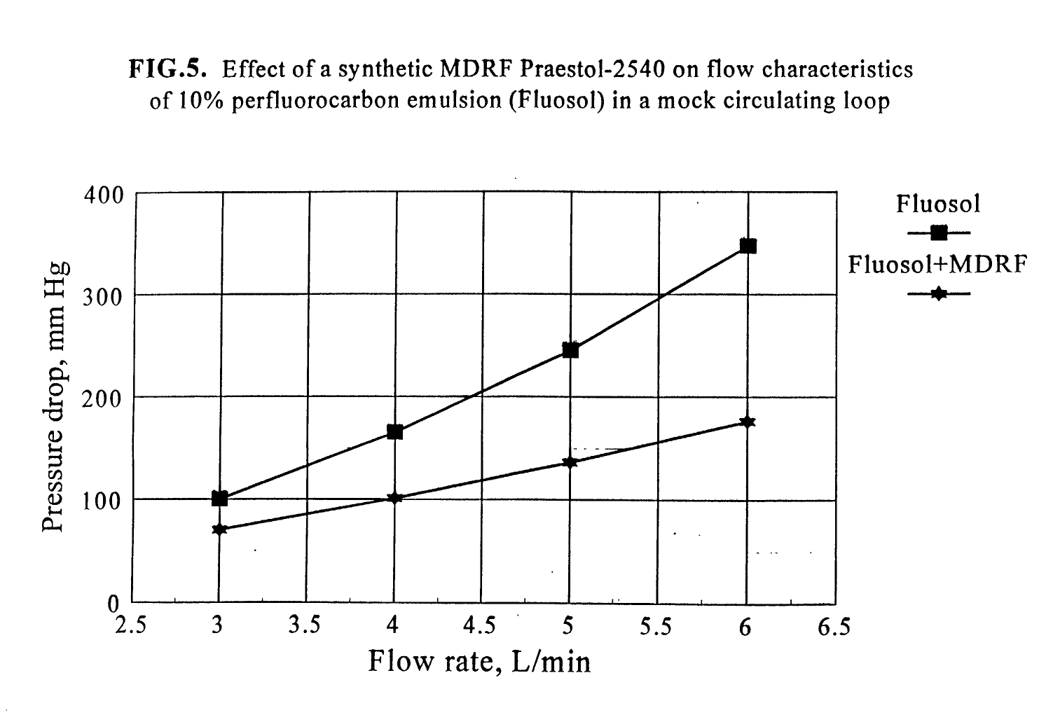

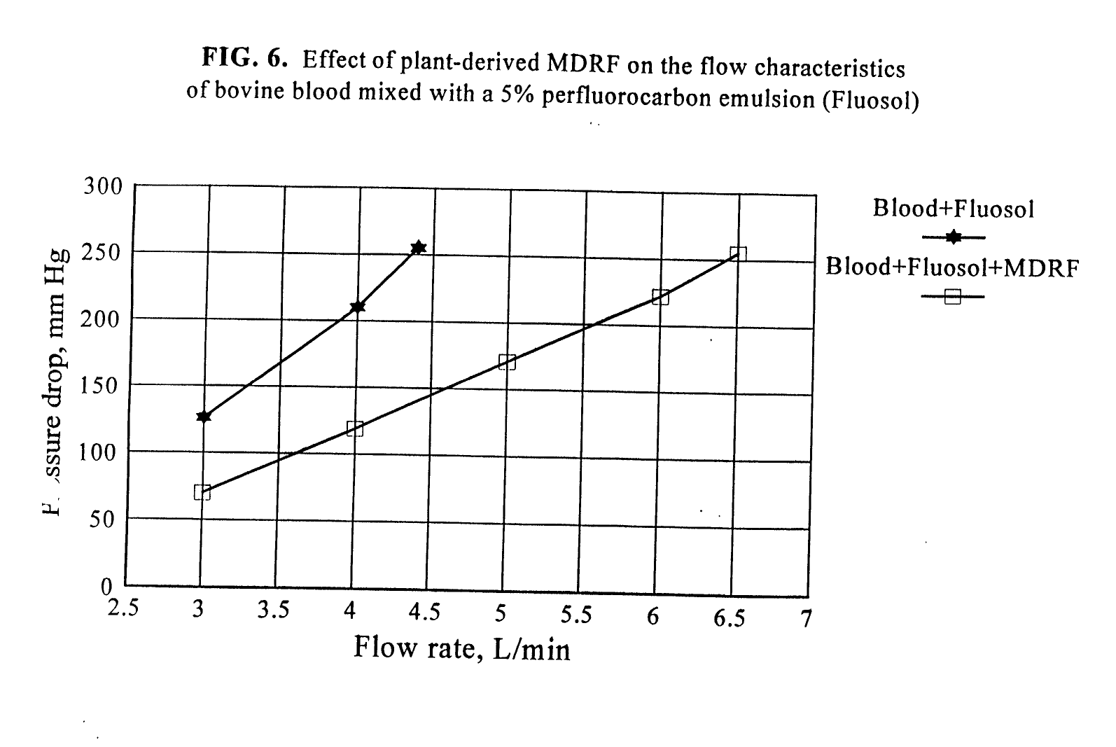

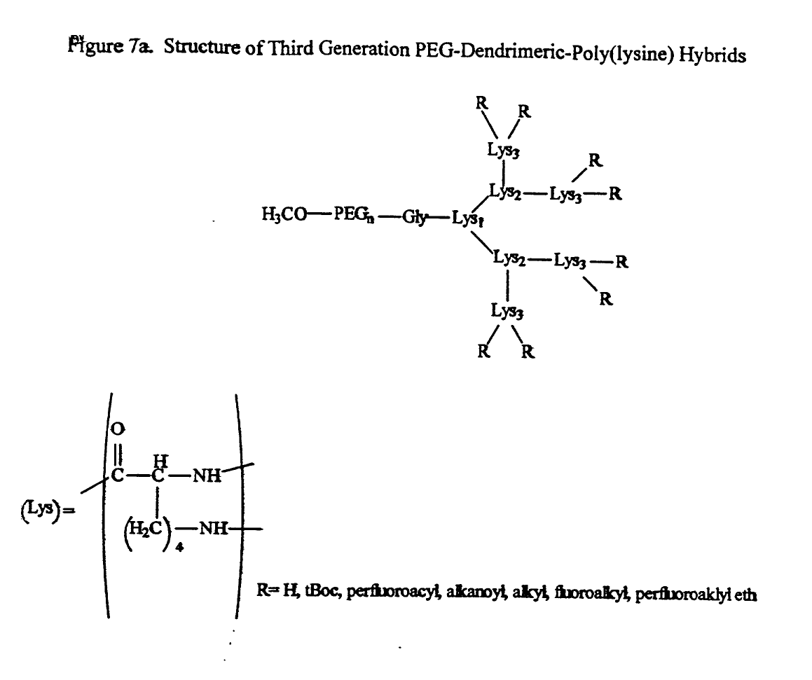

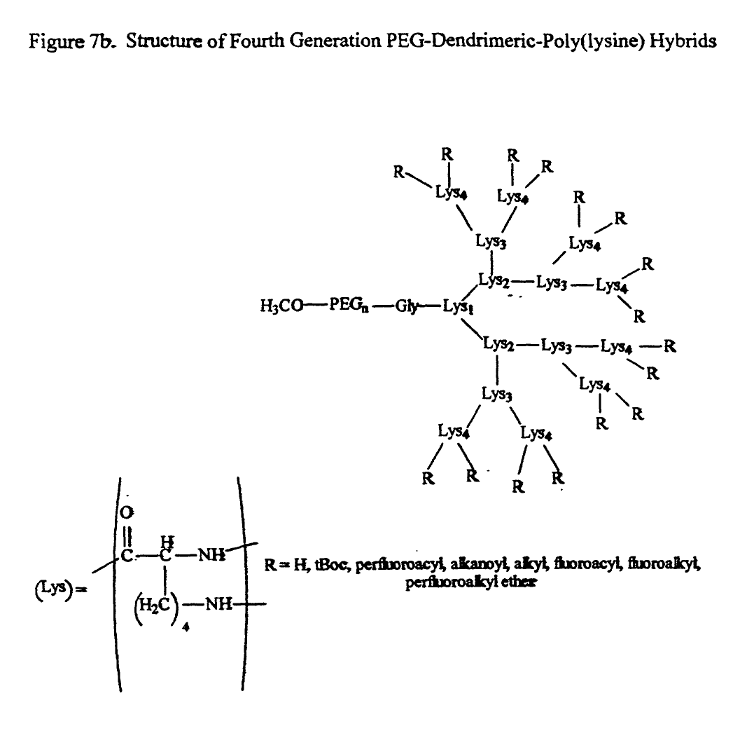

While all effective, pharmaceutically acceptable emulsifiers are contemplated for use within the spirit of this invention, the employment of certain emulsifiers has been found to be particularly useful, with fluorocarbon—based systems. The class of dendritic polymers has been found to be particularly useful. Such polymers are known, per se. For example, it is preferred to employ a third or fourth generation amphiphilic polyethylene glycol (PEG) co-dendritic—polylysine conjugate emusifier system. These emulsifiers have the configuration set forth in FIGS. 7a (third generation) and FIG. 7b (fourth generation). When employed, the emulsifier is preferably present in a concentration of about 0.05 to 5 g/dl of the composition. Third and fourth generation amphiphilic PEG-co-dendritic-polylysines are preferred. For a discussion of their preparation and structure, see Chapman, Hydraamphiphiles: Novel Linear Dendritic Block Copolymer Surfactants, Journal of the American Chemical Society, 1994, 116, 11195-96, incorporated by reference herein. Another preferred embodiment of the invention incorporates an emulsifier comprising a third or fourth generation amphiphilic PEG-co-dendrimeric-polylysine with perfluorocarbon-containing termini. As used herein, “perfluorocarbon-containing termini” includes not only the generally understood moiety, having carbon chains wherein every hydrogen is replaced with fluorine, (i.e. all C—F an no C—H bonds) but also includes carbon chains wherein some hydrogens have not been replaced with fluorines (i.e., combinations of both C—F and C—H bonds).

While the addition of certain drag reducing species to certain taansfusion fluids has been proposed heretofore, the benefits of the present invention have not been made available in the prior art.

U.S. Pat. No. 3,590,124—Hoyt, discloses a composition for injection into the blood system comprising a blood transfusion fluid, and 5 to 100 parts per million by weight of high molecular weight, water soluble polyethylene oxide, polyacrylamide, and linear polysaccharides. Partially hydrolyzed dextran in an isotonic sodium chloride solution, normal physiological saline, and normal liquid human plasma are disclosed as transfusion fluids suitable for use in the invention. An object of the invention is reduction of the turbulent friction properties of the transfusion fluid, and thus reduction of the body pumping requirements for the person receiving the transfusion, however, no efficacy was established for this suggestion.

U.S. Pat. Nos. 4,001,401 and 4,061,736 to Bonsen, Morris and Lover disclose a pharmaceutical composition useful as a blood substitute and blood plasma expander comprising a therapeutically effective amount of cross-inked, stromal-free hemoglobin, soluble in aqueous and physiological fluids, capable of reversibly binding a ligand and having a molecular weight of 64,000 to 1,000,000 D miixed with a pharmaceutically acceptable carrier. The pharmaceutically acceptable carrier is a member selected from the group consisting of poly(ethylene oxide), polyacrylamide, polyvinyl pyrrolidone, polyvinyl alcohol, ethylene oxide-polypropylene glycol condensates and polysaccharides, dextran, gum arabic, plasma proteins, albumin, pectin, fluid gelatin and hydroxyethyl starch as crystalloids and colloid polymeric solutions. There is no detail in the patent on either concentration or molecular weight of the poly(ethylene oxide), polyacrylamide and polysaccharides. They are believed to be used as colloids or crystalloids and not to confer drag reducing effects.

U.S. Pat. No. 4,105,798 in the name of Moore et al., discloses an artificial blood comprising an emulsion of a non-aromatizable perfluorinated material in water, the amount of water being greater than 40% by volume, said emulsion containing a non-toxic emulsifier and a perfluorinated C₉-C₁₈ polycyclic hydrocarbon containing at least two bridgehead carbon atoms linked through a bridge containing at least one carbon atom.

The present invention provides artificial blood fluids having relatively low amounts of perfluorochemical-based, hemoglobin-based or other natural or synthetic oxygen carriers. This is made possible by the addition of microflow drag reducing factors in accordance with the invention. Such materials make artificial blood fluids with the relatively low concentrations of oxygen carriers. This modification gives rise to improved cost and efficacy factors. Thus, effective oxygen transport is achieved without the need for large amounts of oxygen carrier. Untoward side effects of the oxygen carriers, emulsifiers, surfactants and others essential components are, accordingly, minimized as are costs attendant to the manufacture of the artificial blood fluids. Since relatively low concentrations of oxygen carriers and other components (surfactants, emulsifiers, etc) can be used advantageously, it is now possible to provide concentrated fluids. These shelf stable fluids have oxygen carriers present along with microflow drag reducing factors, suspension agents, salts and other components, and may advantageously be included in ratios such that dilution to working blood substitute fluids can easily be accomplished. Thus, such fluid concentrates may be diluted significantly, such as from about 2:1 to about 10:1 or more with sterile carrier, conveniently Ringers lactate, saline or the like, to form a large volume of artificial blood fluid. The concentrated fluids are shelf stable.

For example, working artificial blood fluids are prepared in accordance with this invention comprising from about 1 (or below) to about 5 grams per deciliter of a hemoglobin-based oxygen carrying compound together with from about 0.1 to about 10,000 parts per million, by weight, of microflow drag reducing factor or factors. Any of the hemoglobin-based oxygen carrying compounds and compositions described hereinbefore and others as may be developed hereinafter may be employed in connection with this embodiment of the invention.

It is preferred that the amount of hemoglobin-based oxygen carrier comprise from 2 to about 4 grams per deciliter of the artificial blood fluid, with from 2.5 to about 3.5 grams being more preferred. It is preferred that the microflow drag reducing factor be present in an amount of from about 1 to 1000 ppm, with about 5 to about 500 ppm being more preferred.

The concentrates from which working artificial blood fluids may be reconstituted preferably comprise from about 50 to 99.9% by weight of hemoglobin-based oxygen carrier with a drag reducing agent in an appropriate amount such that when diluted, it is effective for reducing drag in artificial blood fluids. The concentrated fluids may also comprise physiologically acceptable carriers and the like.

### EXAMPLE 1

Praestol™ (Stockhausen, Inc., Greensboro, N.C.), believed to be a cationically modified polypropylene material, was added to circulating artificial blood fluid, 10% perfluorochemical emulsion, Fluosol® (Alpha Therapeutic Corporation, Los Angeles, Calif.). FIG. 5 demonstrates in a model blood vessel system that the addition of only trace amounts of Praestol™ (concentration of 10⁻⁵ g/ml) significantly increased (by up to 50%) the flow rate at the same driving pressure. Further, the required driving pressure was reduced up to 100% at constant flow rate.

### EXAMPLE 2

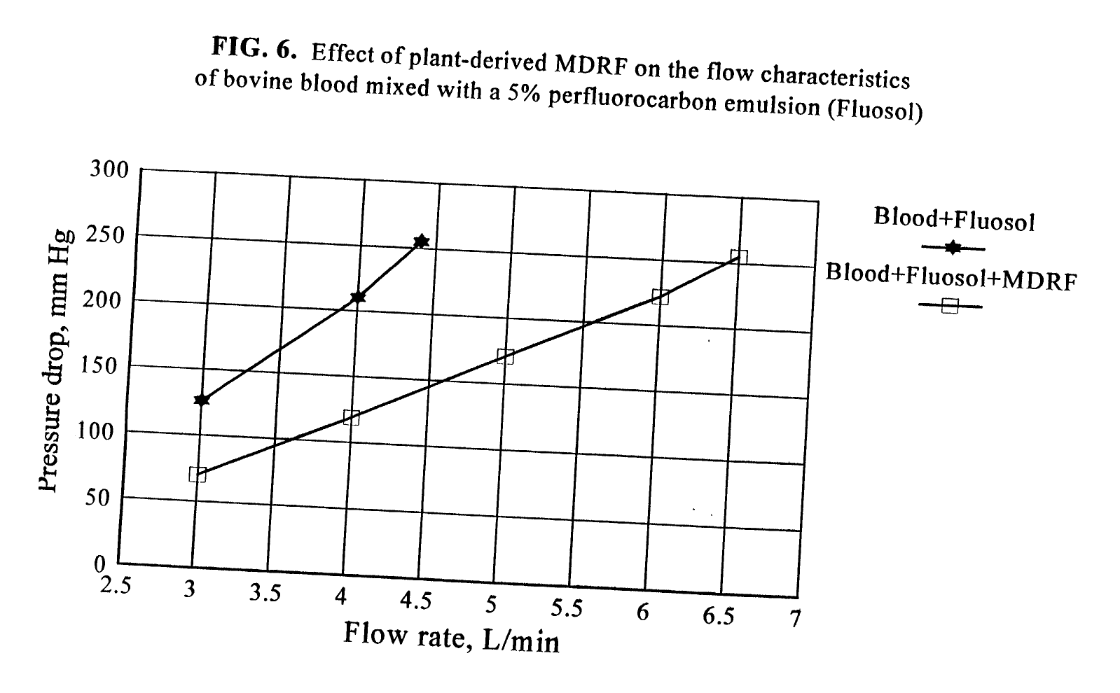

A polysaccharide derived from plant (okra) was added to bovine blood mixed with the 5% perfluorochemical emulsion Fluoso®. FIG. 6 demonstrates in a model blood vessel system under turbulent flow conditions that the addition of this polysaccharide significantly increased (by more than 40%) the blood-Fluosol mixture flow rate at the same driving pressure and reduced driving pressure up to 80% at constant flow rate.

### EXAMPLE 3

Biopolymers derived from human or animal blood plasma and erythrocytes, such as those disclosed in Grigorian, S. S., Kameneva, M. V. et al., Proceedings of the Academy of Sciences of the USSR, Biophysics Section, July-December 1987, 178-179, which are purified, can be added to artificial blood fluids in accordance with this invention. An effect similar to that described in the EXAMPLES 1 and 2 can be achieved.

### EXAMPLE 4

Purified biopolymeric microflow drag reducing factors derived from plants, sea weed, fresh water or marine algae, or bacteria, such as those described in Shenoy A. V., Colloid and Polymer Science, 262, 319-337, (1984), incorporated herein by reference, can be added to an oxygen carrying solution to form artificial blood fluids in accordance with this invention. A physiologically relevant effect similar to that described in the EXAMPLES 1, 2 and 3 can be achieved

### EXAMPLE 5

A plant-derived polysaccharide which meets the definitions of microflow drag reducing factor, was intravenously injected into normal rats. A typical response following such injection is shown on FIG. 1a. The major baseline hemodynamic parameters changed significantly after injection. Tissue perfusion increased from 7.5 tissue perfusion units (TPU) to 33 TPU, systolic blood pressure decreased from 105 mm Hg to 90 mm Hg. Thus the tissue vascular resistance was decreased as much as 5 times. As a control, Sodium Nitroprusside, a powerful clinically-used vasodilator, was intravenously injected into normal rats. A typical response following such injection is shown on FIG. 1b. Tissue perfusion decreased from 6.5 TPU at the baseline to 2.0 TPU, systolic blood pressure decreased from 110 mm Hg to 70 mm Hg. Thus the tissue vascular resistance was increased as much as 2 times. These results demonstrate that this factor very effectively decreased vascular resistance and increased oxygen supply to the tissue. This increase in vascular conductivity was not simply caused by vasodilation.

### EXAMPLE 6

Blood plasma was partially replaced with artificial blood fluid (perfluorocarbon emulsion, PFC, Fluosol-DA, Alpha Therapeutic Corp., Los Angeles, Calif.) during in-vitro experiments using a heart-assist device (a centrifugal pump) and a mock circulatory loop. These experiments showed that the replacement of 20% plasma volume with PFC reduced hemolysis (plasma free Hb released from destroyed red blood cells) by approximately 40% compared to controls. A 20% replacement of plasma vohlme with PFC remarkably improved rheologic properties of human donor blood; in particular low shear blood viscosity and erythrocyte sedimentation rate were reduced, indicating a reduction of erythrocyte aggregation.

### EXAMPLE 7

Red blood cells separated from plasma were suspended in a solution of Polyethylene glycol (PEG, Sigma Chemical Co., Molecular weight of about 15,000-20,000 D) or in solution of dextra (Dextran-40, Sigma Chemical Co., Molecular weight of about 40,000 D). Then, the suspensions were both exposed to simla mechanical strs. Damage to red blood cells suspended in dextran was as much as three times higher than damage to red blood cells suspended in polyethylene glycol solution. The presence of polyethylene glycol in the suspension medium reduced mechanical damage to tile red blood cells. Therefore, the polyethylene glycol can be used in the compositions of the present invention for the protection of blood cells from mechanical damage, as occurs clinically in extracorporeal and implanted heart and lung assist devices, dialysis machines and other blood-wetted artificial organs.

### EXAMPLE 8

20 ounces of cut or chopped frozen okra was acquired from a grocery store and thawed. The okra was mixed with about 1,000 ml of phosphate buffered saline, saline or water in order to extract the mucilaginous portion of the plant. The resulting mixture was mixed for about 2 to about 4 hours, filtered using a nylon straining bag and the resulting filtrate collected. A press was used to squeeze residual liquid from the remaining mixture and the residual liquid collected.

The total collected liquid was centrifuged for about 120 minutes at about 9,000 rpm to remove large particulate matter. The resulting supernatant was filtered using a filter having a pore size of about 100 micrometers to about 0.04 inches in order to remove residual large particulate matter. The resulting filtrate was tested in-vitro for its drag reducing effectiveness using an experimental circulating loop as described supra.

Microflow drag reducing factor was selectively precipitated from the filtrate by the addition of two volumes of 95% ethanol, an aqueous solution of 3.2 molar ammonium sulfate or 100 mg/ml (w/v) of cetylpyridinium chloride. The microflow drag reducing factor precipitate was removed by centrifuging at about 3,600 rpm for about 15 minutes and/or filtration. The microflow drag reducing precipitate was also removed by filtration through a Buchner funnel. The precipitate was washed with about 200 ml of 100% ethanol. The precipitate was dried overnight in a vacuum.

The dried precipitate was dissolved in about 1,000 ml of phosphate buffered saline or saline. The precipitate was resuspended in about 1,000 ml of phosphate buffered saline or saline and slowly stirred at about 4° C. It may take several days to dissolve the precipitate. The resulting solution was dialyzed against phosphate buffered saline or saline where precipitation included the use of salts such as ammonium sulfate or cetylpyridinium chloride. Dialysis was performed in order to remove salt. The resulting solution was centrifuged at about 16,000 rpm for about 120 minutes at about 4° C. The resulting supernatant was collected and tested for drag reducing effectiveness using an experimental circulating loop as described supra.

The resulting supernatant was mixed with two volumes of 95% ethanol in order to precipitate microflow drag reducing factor. The precipitated microflow drag reducing factor was separated from the supernatant by filtration through a Buchner funnel. The precipitated microflow drag reducing factor was washed with about 200 ml of 100% ethanol. The precipitated microflow drag reducing factor was dried overnight in a vacuum.

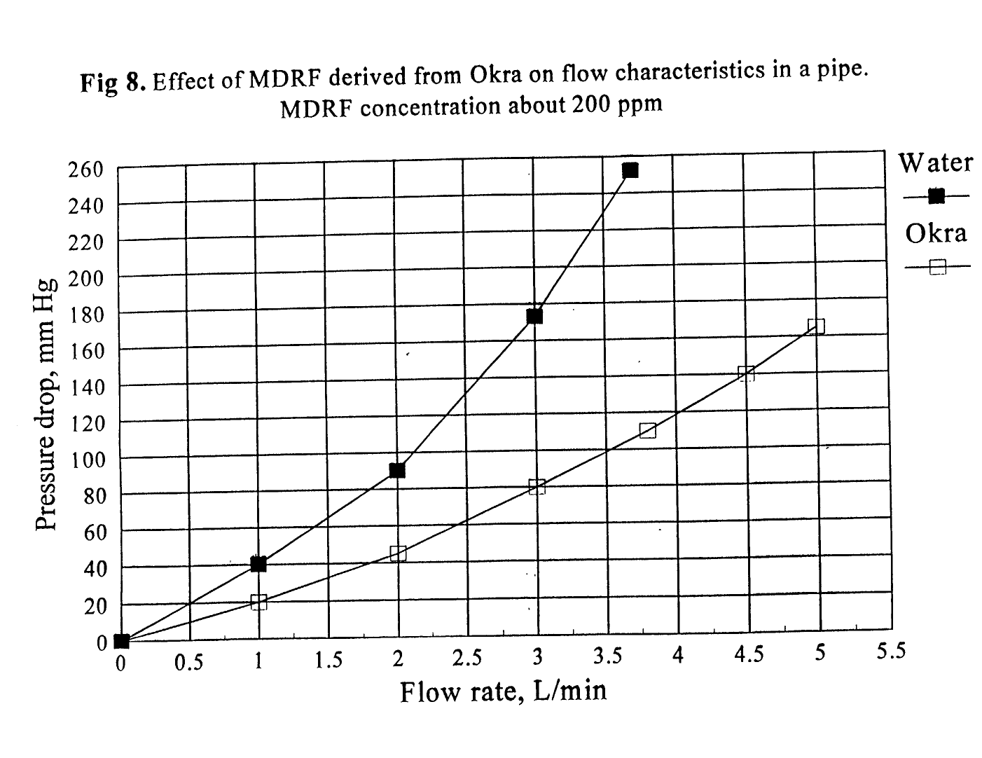

The dried microflow drag reducing factor was dissolved in about 1,000 ml of phosphate buffered saline or saline. The dried microflow drag reducing factor was resuspended in about 1,000 ml of phosphate buffered saline or saline and slowly stirred at about 4° C. The resulting solution was centrifuged at about 16,000 rpm for about 120 minutes at about 4° C. The resulting supernatant was collected and tested for drag reducing effectiveness using an experimental circulating loop as described supra. With reference to FIG. 8, the flow and pressure characteristics of saline flow in an experimental circulating loop were measured before and after the addition of the okra-derived microflow drag reducing factor. A pressure gradient reduction of up to about 100 percent was achieved by the addition of microflow drag reducing factor having a concentration from about 10 to about 100 ppm to saline flowing at a rate of about 3-4 liters per minute.

The resulting microflow drag reducing factor was purified using enzymes. The microflow drag reducing factor was further purified using tangential flow systems, microfiltration systems, dialysis, size exclusion chromatography and/or ion exchange chromatography. The microflow drag reducing factor was tested for drag reducing effectiveness in vitro using an experimental circulation loop as described supra. The microflow drag reducing factor was storied and tested in an animal body. The microflow drag reducing factor was chemically modified as discussed supra.

### EXAMPLE 9

About 45 to 50 grams of leaves were removed from the aloe vera plant and washed in water to remove dirt and excess particulate matter. All attempts were taken to keep the aloe vera-derived microflow drag reducing factor cold and in a sealed environment. The leaves were sliced open lengthwise and the exposed gel scraped from the interior of the leaves. The gel was then mixed with about 100 ml of phosphate buffered saline at a pH of about 7.4. This mixture was filtered through several layers of cheesecloth and the resulting filtrate collected and stirred for about 30 minutes at about 4° C.

The aloe vera extract was then centrifuged at about 16,500 rpm (39,000 g) for about 60 minutes at about 4° C. The resulting supernatant was collected and tested for drag reducing effectiveness in vitro using an experimental circulation loop as described supra.

The microflow drag reducing factors were then selectively precipitated from the supernatant. Two volumes of 95% ethanol, aqueous solutions of 3.2 molar ammonium sulfate or 100 mg/ml (w/v) cetylpyridinium chloride were added to the supernatant. Microflow drag reducing factors were separated from the solution by centrifugation at about 3,600 rpm for about 15 minutes and/or filtration. The precipitate was removed by filtration through a Buchner fennel. The precipitate was washed with 200 ml of 100% ethanol. The precipitate was dried overnight in a vacuum.

The dried precipitate was then dissolved in a solvent. The precipitate was resuspended in about 100 ml of phosphate buffered saline and slowly stirred at about 4° C. Several days may be required to dissolve the precipitate. The resulting solution was centriged at about 16,000 rpm (39,000 g) for about 120 minutes at about 4° C. The resulting supernatant was dialyzed against phosphate buffered saline where precipitation included the use of salt such as ammonium sulfate or cetylpyridinium chloride. The supernatant was collected and tested for drag reducing effectiveness using an experimental circulating loop as described supra.

The supernatant was subjected to an additional organic solvent wash. The supernatant was mixed with two volumes of 95% ethanol to form a precipitate. A Buchner fimnel was used to collect the precipitate. The precipitate was washed with 200 ml of 100% ethanol. The precipitate was dried overnight in a vacuum.

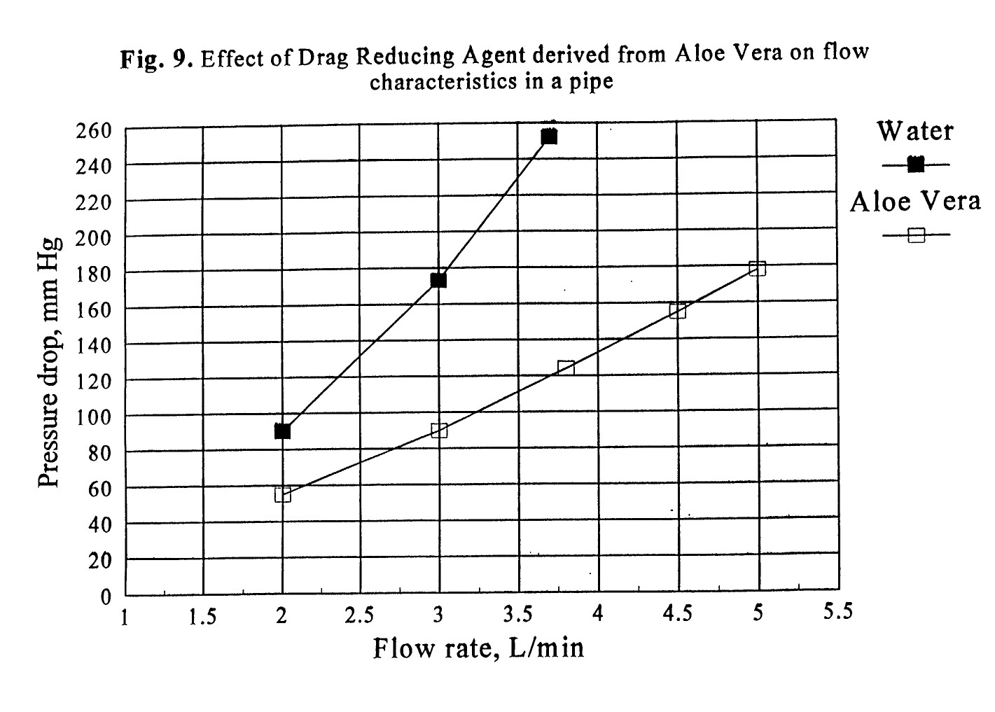

The precipitate was then dissolved in a solvent. The precipitate was resuspended in about 1,000 ml of phosphate buffered saline or saline and stirred slowly at about 4° C. The resulting solution was centrifuged at about 16,000 rpm for about 120 minutes at about 4° C. The resulting supernatant was collected and tested for drag reducing effectiveness using an experimental circulating loop as described supra. With reference to FIG. 9, the flow and pressure characteristics of saline flowing in an experimental circulating loop were measured before and after the addition of the aloe vera-derived microflow drag reducing factor. A pressure gradient reduction of up to about 100 percent was achieved by adding microflow drag reducing factor having a concentration from about 10 to about 100 ppm to saline flowing at a rate of about 3-4 liters per minute.

The resulting nicroflow drag reducing factor was then purified using enzymes (proteases, DNAases, RNAases). The microflow drag reducing factor was further purified using tangential flow systems, microfiltration systems, dialysis, size exclusion chromatography and/or ion exchange chromatography. The microflow drag reducing factor was then tested for drag reducing effectiveness in vitro using an experimental circulation loop as described supra. The microflow drag reducing factor was then sterilized and tested in an animal body. The microflow drag reducing factor was then chemically modified as discussed supra.

### EXAMPLE 10

Microflow drag reducing factor was extracted from Natto. Natto was soaked in a solvent of phosphate buffered saline, saline or water and agitated in order to extract the microflow drag reducing factor. The resulting suspension was passed through a series of filters of about 2 mm to about 20 microns after soaking for at least about 2 hours in order to remove soybeans and large particulate matter. The suspension was also passed through a cheesecloth in order to remove large particles. The resulting filtrate was centrifuged at low speed of about 2,000 to about 4,000 rpm for about 30 to about 120 minutes. The resulting supernatant was carefully removed so as not to disturb the pellet. The supernatant was centrifuged at high speed of about 7,000 to about 20,000 rpm for about 30 to about 120 minutes in order to remove additional particulate matter. Centrifugation was performed several times until a clear supernatant was obtained. The clear supernatant was tested for drag reducing effectiveness in vitro using an experimental circulation loop as described supra.

The clear supernatant was subjected to dialysis using membranes having an about 7,000 to about 2,000,000 molecular weight cutoff rating for about 2 to about 48 hours in order to isolate microflow drag reducing factor from low molecular weight impurities. Efficiency of the dialysis procedure may be measured by testing the dialysis buffer for changes in viscosity at the end of the procedure. The microflow drag reducing factor was further purified by treatment with proteses (trypsin, chymotrypsin), DNAases and/or RNAases. The microflow drag reducing factor was then subjected to dialysis in order to remove the enzymes and digested materials. The microflow drag reducing factor was further purified by tangential flow systems, microfiltration systems, size exclusion chromatography (Sepharose, Sigma Chemical Company) and/or ion exchange chromatography. The microflow drag reducing factor was tested for drag reducing effectiveness in vitro using an experimental circulation loop as described supra. The microflow drag reducing factor was sterilized and tested for effectiveness in an animal body. The microflow drag reducing factor was then chemically modified as described supra. The microflow drag reducing factor was lyophilieed for storage or precipitated with organic solvents and/or aqueous salt solutions, as described supra.

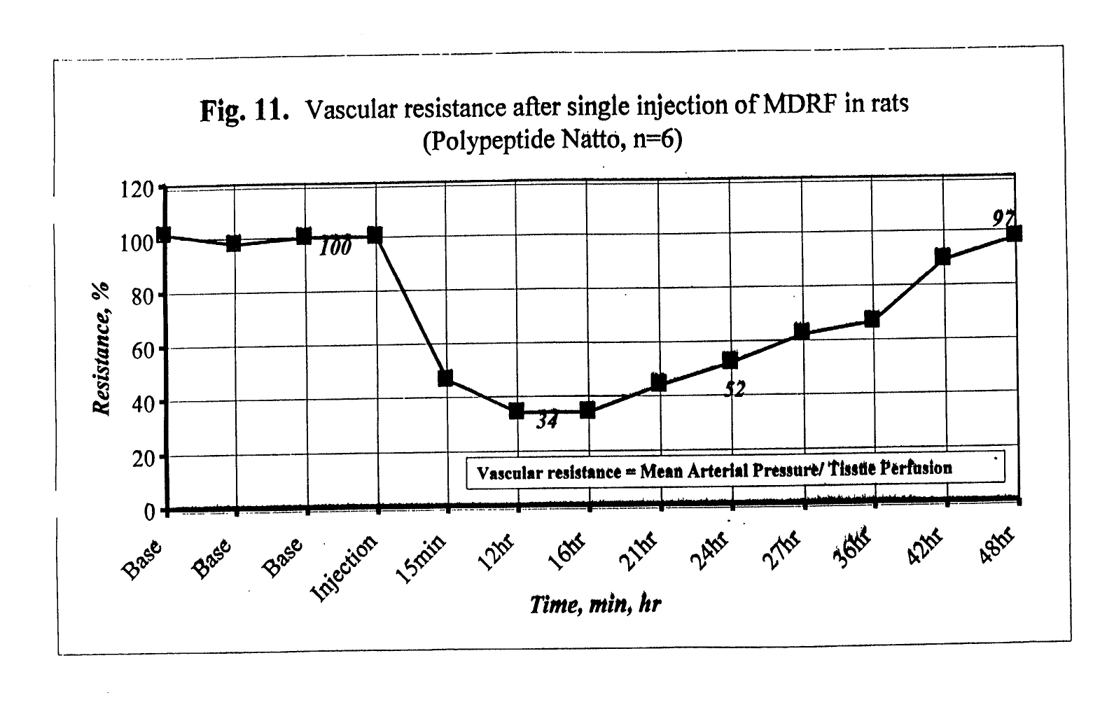

With reference to FIG. 11, the results of six experiments injecting normal rats with Natto derived microflow drag reducing factor prepared according to this Example may be described. The Natto-derived microflow drag reducing factor prepared according to this Example was preliminary tested in vitro for drag reducing effectiveness using the experimental circulating loop as described supra. Rats were then injected intravenously with Natto-derived microflow drag reducing factor prepared according to this Example after the measurement of base hemodynamic parameters. Blood pressure was measured non-invasively from the tail. Tissue perfusion (cheek mucous) was monitored using a laser-Doppler flowmeter. All rats were lightly anesthetized with Ketamine at each point of measurement. Again with reference to FIG. 11, it can be seen that vascular resistance decreased to 45% of the base level measurement immediately after injection of the Natto-derived microflow drag reducing factor, and remained significantly lower than the base level for about 36 hours. Vascular resistance returned to base level about 48 hours after injection. Vascular resistance was calculated as the ratio of arterial blood pressure to tissue perfusion. Each point on the graph represents an average of the measurements taken in the six tests.

### EXAMPLE 11

Hemorrhage was induced in Ketamine/Xylazine-anesthetized rats by bleeding at a rate of about 0.5 to about 1 millileter per minute until a mean arterial pressure of 25 mm Hg was achieved. Hemodynamic parameters including mean arterial pressure, pulse pressure, heart rate and tissue perfusion were monitored. Arterial blood pressure was measured and recorded by catheterizing the left carotid artery and employing a cardiovascular patient monitor and pressure transducers. A laser-Doppler flowmeter was used to monitor tissue perfusion in cheek mucous, tongue, skin surface and muscle mass. Blood pressure and tissue perfusion levels were recorded with a Win Daq/Pro data acquisition system. Blood samples of about 0.5 ml were withdrawn from the jugular vein in order to record hematologic parameters including blood hemoglobin, hematocrit and pH. These blood samples were withdrawn at the inception as well as at various intervals during the experiment.

The experiment was begun by recording base hemodynamic parameters for about 30 minutes, withdrawing a base blood sample and inducing severe hemorrhage. Severe hemorrhage was induced by using a syringe pump to slowly withdraw about 50% of circulating blood volume from the tail vein. Hemorrhage was stopped when mean arterial pressure decreased to about 20 to about 25 mm Hg. Tissue perfusion had decreased to about 0.5 to about 1.5 Tissue Perfusion Units (TPU) when hemorrhagic shock (peripheral circulatory failure) developed.

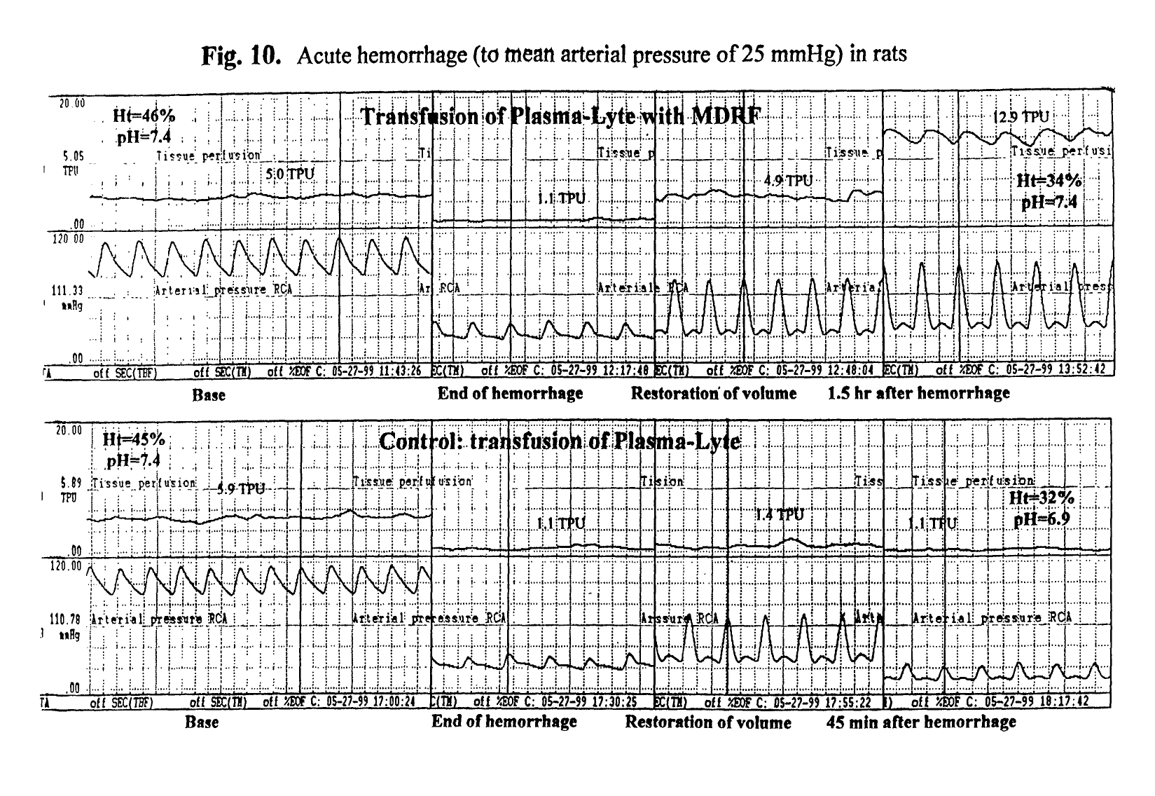

About 5 minutes after discontinuing blood withdrawal, control animals were transfuse with Plasma-Lyte while test animals were transfused with Plasma-Lyte and about 2 ppm of microflow drag reducing factor. With reference to FIG. 10, the results of one such experiment may be described. The transfusion of Plasma-Lyte in the control animals did not produce any improvements in hemodynamic parameters and the control animals died about 45 minutes after beginning blood volume restoration. The control animals experienced severe acidosis as indicated by a blood pH of about 6.9. In contrast, the hemodynamic parameters of the test animals improved immediately after transfusion with Plasma-Lyte and microflow drag reducing factor. Tissue perfusion was quickly restored and even exceeded the base level. Hemodynamic parameters remained stable after the restoration of blood volume for the entire observation period. In some experiments, with reference to FIG. 4, hemodynamic parameters remained stable for about 2 to about 3 hours after the restoration of blood volume. The acid-base equilibrium, impaired after hemorrhage, also normalized after the restoration of blood volume.

Compositions of the present invention can also be used in powerful methods for treating impaired microcirculation in patients having severe blood circulatory disorders. By administering the composition of the present invention to such a patient, generally intravenously, the composition increases the fluidity of the patient's blood, thereby improving microcirculatory flow and tissue perfusion in the patient. This improved microcirculation can be achieved by administering a composition including the MDRF by itself, the MDRF with a pharmaceutically acceptable carrier, or the MDRF with the oxygen carrying compound, or with both the oxygen carrying compound and an emulsifier as disclosed herein. Such methods may treat, for example, impaired microcirculation in patients suffering from one or more conditions such as diabetes, acute myocardial infarction, acute transient cerebral ischemic attack, ischemic heart disease, sickle cell disease, atherosclerosis, and other known blood circulatory disorders.

The present invention also improves the extracorporeal survivability of organs for transplantation. Perfusion of such organs with artificial blood fluids of this invention improves oxygenation with minimal damage to the tissues involved.

Numerous modifications and variations of the present invention are expected to occur to those skilled in the art upon consideration of the above description. Although the invention has been described herein with references to certain preferred embodiments and in the general context of artificial blood composition, the microflow drag reducing factors of the present invention are equally applicable to other uses in which increased microcirculatory flow rates are desired, but with no increase in pressure drop across the system. These and all other improvements, modifications, and variations to the spirit of the invention are intended to fall within its scope, as set forth in the following claims, including the full range of equivalents thereof.

What is claimed:

1. An artificial blood fluid comprising a physiologically acceptable carrier, at least one perfluorocarbon—based oxygen carrying compound, and from about 0.1 ppm to about 10,000 ppm of at least one microflow drag reducing factor.

2. The artificial blood fluid of claim 1 which is sterile, non-pyrogenic and shelf stable.

3. The artificial blood fluid of claim 1 wherein said perfluorocarbon—based oxygen carrying compound is present in a concentration of from about 1 to about 20 grams per deciliter of fluid.

4. The artificial blood fluid of claim 1 wherein said perfluorocarbon—based oxygen carrying compound is present in a concentration of from about 2.5 to about 15 grams per deciliter of fluid.

5. The artificial blood fluid of claim 1 wherein said pertluorocarbon—based oxygen carrying compound is present in a concentration of from about 5 to about 10 gram per deciliter of fluid.

6. The artificial blood fluid of claim 1 wherein said perfluorocarbon—based oxygen carrying compound is present in a concentration of from about 1 to about 10 grams per deciliter of fluid.

7. The artificial blood fluid of claim 1 wherein the microflow drag reducing factor is present in an amount between about 1 and about 1,000 ppm.

8. The artificial blood fluid of claim 1 wherein the microflow drag reducing factor is present in an amount between about 5 and about 500 ppm.

9. The artificial blood of claim 1 wherein the microflow drag reducing factor is derived from Natto.

10. The artificial blood of claim 1 wherein the microflow drag reducing factor is derived from the Lily plant family.

11. The artificial blood of claim 10 wherein the microflow drag reducing factor is derived from aloe vera.

12. The artificial blood of claim 1 wherein the microflow drag reducing factor is derived from the Mallow plant family.

13. The artificial blood of claim 12 wherein the microflow drag reducing factor is derived from okra.

14. The artificial blood fluid of claim 1 further comprising at least one emulsifier.

15. The artificial blood fluid of claim 14 wherein said emulsifier has a concentration of from about 0.05 to about 5 grams per deciliter.

16. The artificial blood fluid of claim 14 wherein said emulsifier has a concentration of from about 0.1 to about 3 grams per deciliter.

17. The artificial blood fluid of claim 14 wherein said emulsifier has a concentration of from about 0.5 to about 2 grams per deciliter.

18. The artificial blood of claim 14 wherein said emulsifier is a third generation amphiphilic PEG-co-dendritic-polylysine with termini conjugated with perfluorocarbon-containing moieties.

19. The artificial blood of claim 14 wherein said emulsifier is a fourth generation amphiphilic PEG-co-dendritic-polylysine with termini conjugated perfluorocarbon-containing moieties.

20. A concentrate for use in the formulation of an artificial blood fluid comprising from about 50% to about 99.9% by weight of perfluorocarbon—based oxygen carrying compound and at least one microflow drag reducing factor.

21. The concentrate of claim 20 further comprising a physiologically acceptable carrier.

22. The concentrate of claim 20 further comprising at least one emulsifier.

23. The concentrate of claim 20 which, when blended with a physiologically acceptable carrier provides an artificial blood fluid.

24. The concentrate of claim 20 wherein the microflow drag reducing factor is derived from Natto.

25. The concentrate of claim 20 wherein the microflow drag reducing factor is derived from the Lily plant family.

26. The concentrate of claim 25 wherein the microflow drag reducing factor is derived from aloe vera.

27. The concentrate of claim 20 wherein the microflow drag reducing factor is derived from the Mallow plant family.

28. The concentrate of claim 27 wherein the microflow drag reducing factor is derived from okra.

29. An artificial blood fluid comprising a physiologically acceptable carrier, from about 1 to about 9 grams per deciliter of at least one hemoglobin—based oxygen carrying compound, and from about 0.1 ppm to about 10,000 ppm of at least one microflow drag reducing factor.

30. The artificial blood fluid of claim 29 which is sterile, non-pyrogenic and shelf stable.

31. The artificial blood fluid of claim 29 wherein said hemoglobin—based oxygen carrying compound is present in a concentration of from about 2 to about 8 grams per deciliter of fluid.

32. The artificial blood fluid of claim 29 wherein said hemoglobin—based oxygen carrying compound is present in a concentration of from about 2.5 to about 5 grams per deciliter of fluid.

33. The artificial blood fluid of claim 29 wherein said microflow drag reducing factor is present in an amount between about 1 and about 1000 ppm.

34. The artificial blood fluid of claim 29 wherein said microflow drag reducing factor is present in an amount between about 5 and about 500 ppm.

35. The artificial blood of claim 29 wherein the microflow drag reducing factor is derived from Natto.

36. The artificial blood of claim 29 wherein the microflow drag reducing factor is derived from the Lily plant family.

37. The artificial blood of claim 36 wherein the microflow drag reducing factor is derived from aloe vera.

38. The artificial blood of claim 29 wherein the microflow drag reducing factor is derived from the Mallow plant family.

39. The artificial blood of claim 38 wherein the microflow drag reducing factor is derived from okra.

40. A concentrate for use in the formulation of an artificial blood fluid comprising from about 50% to about 99.9% by weight of hemoglobin-based oxygen carrying compound and at least one microflow drag reducing factor.

41. The concentrate of claim 40 further comprising a physiologically acceptable carrier.

42. The concentrate of claim 40 which, when blended with a physiologically acceptable carrier provides an artificial blood fluid.

43. The concentrate of claim 40 wherein the microflow drag reducing factor is derived from Natto.

44. The artificial blood of claim 40 wherein the microflow drag reducing factor is derived from the Lily plant family.

45. The artificial blood of claim 44 wherein the microflow drag reducing factor is derived from aloe vera.

46. The artificial blood of claim 40 wherein the microflow drag reducing factor is derived from the Mallow plant family.

47. The artificial blood of claim 46 wherein the microflow drag reducing factor is derived from okra.

48. An artificial blood fluid comprising a physiologically acceptable carrier, from about 0.1 to about 5 grams per deciliter of at least one synthetic or naturally-occurring oxygen carrying compound, and from 0.1 ppm to about 10,000 ppm by weight of the fluid, of at least one microflow drag reducing factor.

49. The artificial blood fluid of claim 48 further comprising a polyethylene glycol.

50. The artificial blood fluid of claim 48 wherein said synthetic oxygen carrying compound is present in a concentration of from about 1 to about 4 grams per deciliter of fluid.

51. The artificial blood fluid of claim 48 wherein said synthetic oxygen carrying compound is present in a concentration of from about 2 to about 3.5 grams per deciliter of fluid.

52. The artificial blood fluid of claim 48 wherein said microflow drag reducing factor is present in an amount between about 1 and about 1000 ppm.

53. The artificial blood fluid of claim 48 wherein said microflow drag reducing factor is present in an amount between 5 and about 500 ppm.

54. The artificial blood of claim 48 wherein the microflow drag reducing factor is derived from Natto.

55. The artificial blood of claim 48 wherein the microflow drag reducing factor is derived from the Lily plant family.

56. The artificial blood of claim 55 wherein the microflow drag reducing factor is derived from aloe vera.

57. The artificial blood of claim 48 wherein the microflow drag reducing factor is derived from the Mallow plant family.

58. The artificial blood of claim 57 wherein the microflow drag reducing factor is derived from okra.

59. The artificial blood fluid of claim 48 winder comprising at least one emulsifier.

60. The artificial blood fluid of claim 48 wherein said emulsifier has a concentration of from about 0.05 to about 5 grams per deciliter.

61. The artificial blood fluid of claim 48 wherein said emulsifier is a third generation amphiphilic PEG-co-dendrimeric-polylysine with termini conjugated perfluorocarbon containing moieties.

62. The artificial blood fluid of claim 48 wherein said emulsifier is a fourth generation amphiphilic PEG-co-dendritic-polylysine with termini conjugated solution.

63. A concentrate for use in the formulation of an artificial blood fluid comprising from about 50% to about 99.9% by weight of at least one synthetic or naturally-occurring oxygen carrying compound and at least one microflow drag reducing factor.

64. The concentrate of claim 63 further comprising a physiologically acceptable carrier.

65. The concentrate of claim 63 further comprising at least one emulsifier.

66. The concentrate of claim 63 wherein the microflow drag reducing factor is derived from Natto.

67. The concentrate of claim 63 wherein the microflow drag reducing factor is derived from the Lily plant family.

68. The concentrate of claim 67 wherein the microflow drag reducing factor is derived from aloe vera.

69. The concentrate of claim 63 wherein the microflow drag reducing factor is derived from the Mallow plant family.

70. The concentrate of claim 69 wherein the microflow drag reducing factor is derived from okra.

71. The concentrate of claim 63 which, when blended with a physiologically acceptable carrier, and optionally emulsified, provides an artificial blood fluid.

72. A fluid comprising a physiologically acceptable carrier and about 0.1 ppm to about 10,000 ppm of at least one microflow drag reducing factor.

73. The fluid of claim 72 wherein the microflow drag reducing factor is present in an amount between about 1 and about 1,000 ppm.

74. The fluid of claim 72 wherein the microflow drag reducing factor is present in an amount between 5 and about 500 ppm.

75. The fluid of claim 72 wherein the microflow drag reducing factor is derived from Natto.

76. The fluid of claim 72 wherein the microflow drag reducing factor is derived from the Lily plant family.

77. The fluid of claim 76 wherein the microflow drag reducing factor is derived from aloe vera.

78. The fluid of claim 72 wherein the microflow drag reducing factor is derived from the Mallow plant family.

79. The fluid of claim 78 wherein the microflow drag reducing factor is derived from okra.

80. A method for the improvement of impaired microcirculation in a mammal comprising adding to the blood of said mammal the fluid of claim 1, 29 or 72.

81. The method of claim 80 wherein said impaired microcirculation is associated with hemorrhage, severe trauma, ischemic heart disease, diabetes, acute myocardial infarction, acute transient cerebral ischemic attack, sickle cell disease or atherosclerosis.

82. A method for decreasing blood pressure in a hypertensive patient, wherein circulatory resistance is reduced, without vasodilatation, and while maintaining or increasing microcirculatory blood flow, comprising adding to the blood of said patient an artificial blood fluid of claim 1 or 29.

83. A method for decreasing blood pressure in a patient, wherein circulatory resistance is reduced, without vasodilation, and while maintaining or increasing microcirculatory blood flow comprising adding to the blood of said patient an effective amount of a microflow drag reducing factor.

84. The method of claim 83 wherein said microflow drag reducing factor is administered in a pharmaceutically acceptable carrier or diluent.

85. A method for increasing peripheral blood flow in a patient without increasing blood pressure or vasodilation comprising adding to the blood of said patient an effective amount of a microflow drag reducing factor.

86. The method of claim 85 wherein said microflow drag reducing factor is administered in a pharmaceutically acceptable carrier or diluent.

87. A method for the preservation of an isolated organ comprising perfusing said organ with the artificial blood fluid of claim 1 or 29.

88. A method for protecting blood cells from mechanical damage during extracorporeal manipulation comprising contacting the cells with the artificial blood fluid of claim 1, 29 or 49.

89. A method for increasing the effectiveness of drug delivery to a tissue comprising coadministering the drug with a microflow drag reducing factor.

90. A method for increasing the effectiveness of drug delivery to a tissue comprising coadministering the drug with the fluid of claims 1, 29 or 72.
